(19) United States
(12) Patent Application Publication
(10) Pub. No.: US 2025/0208143 A1
(43) Pub. Date: Jun. 26, 2025

(54) p53 FRAGMENTS AS MARKERS FOR DIAGNOSIS AND PROGNOSIS OF NEURODEGENERATIVE DISEASE STATES

(71) Applicant: Diadem SpA, Milan (IT)

(72) Inventors: Paul Kinnon, Milan (IT); Simona Piccirella, Milan (IT); Donald J. Daley, Milan (IT)

(21) Appl. No.: 18/986,241

(22) Filed: Dec. 18, 2024

Related U.S. Application Data
(60) Provisional application No. 63/614,107, filed on Dec. 22, 2023.

(51) Int. Cl. G01N 33/68; C07K 16/30; C12Q 1/37; G01N 33/537

(52) U.S. Cl. CPC G01N 33/6848; C07K 16/30; C12Q 1/37; G01N 33/537; G01N 33/6896; C07K 2317/565; G01N 2333/4748; G01N 2333/96433; G01N 2333/976; G01N 2800/2814; G01N 2800/52

(57) ABSTRACT

Disclosed are fragments of p53 peptide (P1) and their use in the diagnosis and/or prognosis of Alzheimer's disease (AD) in a biological sample. The invention provides a method based on mass spectrometry analysis for the diagnosis of Alzheimer's disease at the pre-clinical and prodromal stages of the disease and for the prognosis of cognitive decline in a subject, by quantitating the levels of one or more p53 peptide fragments in a biological sample of a subject.

### CROSS-REFERENCE TO RELATED APPLICATIONS

This application claims priority to U.S. Provisional Patent Application No. 63/614,107 filed on Dec. 22, 2023, the content of which is herein incorporated by reference in its entirety.

### TECHNICAL FIELD

The technical field is detection of peptide and peptide fragments for disease diagnosis and/or prognosis.

### SEQUENCE LISTING

This application contains a Sequence Listing which has been submitted electronically as a WIPO Standard ST.26 XML file via Patent Center, created on Dec. 18, 2024, is entitled “121689-10603.xml” and is 26.4 KB in size. The sequence listing is incorporated herein by reference in its entirety.

### BACKGROUND

PCT/EP2015/072094 discloses the development of a new conformational specific anti-U-p53 antibody named 2D3A8, that binds to an epitope (aa 282-297), accessible only when p53 loses its wildtype conformation and becomes an unfolded phenotype. The conformationally altered isoform or unfolded phenotype of p53 protein is found in subjects afflicted with or prone to develop a neurodegenerative disease, for example, Alzheimer's disease (AD) or cognitive impairment related to ageing.

PCT/IB2019/051785 discloses a method of identifying and quantifying p53 peptides in a subject plasma sample using mass spectrometry analysis.

### SUMMARY

The invention relates to the detection of one or more fragments of p53 peptide- P1 (TEEENLR). The invention also relates to detection of one or more fragments of peptide P1 for the diagnosis and/or prognosis of neurological diseases such as Alzheimer's disease (AD) or dementia in a biological sample of a subject.

In one aspect, the invention provides a method of quantifying an isoform of p53 protein in a biological sample of a subject, the isoform being conformationally altered with respect to the wild-type p53 protein, the method comprising the steps of:
contacting the biological sample or a portion thereof of the subject with an anti-p53 antibody comprising heavy chain CDR sequences (CDR1 (SEQ ID NO:8), CDR2 (SEQ ID NO: 9) and CDR3 (SEQ ID NO: 10)) and light chain CDR sequences (CDR1 (SEQ ID NO:11), CDR2 (SEQ ID NO: 12) and CDR3 (SEQ ID NO: 13)) in a reaction mixture to form an immunocomplex comprising the p53 isoform and the antibody;
eluting p53 isoform to provide eluted p53 isoform;
subjecting the eluted p53 isoform or a portion thereof to enzymatic digestion, thereby to generate a composition comprising one or more proteolytic peptides comprising P1 peptide (TEEENLR, SEQ ID NO: 1);
quantifying an amount of the P1 peptide in the composition or a portion thereof by mass spectrometry, wherein the quantifying comprises determining the intensity of mass/charge ratio corresponding to at least one fragment of the P1 peptide to provide a quantified amount of P1 peptide,
wherein a mass to charge ratio (m/z) of 445 identifies P1 peptide, and wherein a mass to charge ratio selected from the group consisting of: m/z 660, m/z 231, m/z 531, m/z 402, m/z 288, and m/z 358 identifies a fragment of P1 peptide:
wherein the m/z 660 identifies a P1 fragment consisting of amino acids EENLR,
the m/z 231 identifies a P1 fragment consisting of amino acids TE,
the m/z 531 identifies a P1 fragment consisting of amino acids ENLR,
the m/z 402 identifies a P1 fragment consisting of amino acids NLR,
the m/z 288 identifies a P1 fragment consisting of amino acids LR, and
the m/z 358 identifies a P1 fragment consisting of amino acids LR doubly charged; and the quantified amount of the P1 peptide is proportionate to the amount of the p53 isoform in the sample or portion of the sample.

In another aspect, the invention provides a method to identify in a subject the presence of Alzheimer's disease or prognosis of cognitive decline leading to dementia, the method comprising the steps of:
providing a biological sample of the subject;
contacting the sample or a portion thereof with an antibody that binds an isoform of p53 antibody that is conformationally altered with respect to the wild-type p53 and comprising heavy chain CDR sequences (CDR1 (SEQ ID NO:8), CDR2 (SEQ ID NO: 9) and CDR3 (SEQ ID NO: 10)) and light chain CDR sequences (CDR1 (SEQ ID NO:11), CDR2 (SEQ ID NO: 12) and CDR3 (SEQ ID NO: 13)), wherein the presence of the isoform of p53 protein in the sample permits formation of an immunocomplex comprising the p53 isoform and the antibody;
eluting the p53 isoform to provide eluted p53 isoform;
subjecting the eluted p53 isoform or a portion thereof to enzymatic digestion, thereby to generate a composition comprising one or more proteolytic peptides comprising P1 peptide (TEEENLR, SEQ ID NO: 1);
quantifying an amount of the P1 peptide in the composition or a portion thereof by mass spectrometry, wherein the quantifying comprises determining the intensity of mass/charge ratio corresponding to at least one fragment of the P1 peptide to provide the quantified amount of P1 peptide,
wherein a mass to charge ratio (m/z) of 445 identifies P1 peptide, and wherein a mass to charge ratio selected from the group consisting of: m/z 660, m/z 231, m/z 531, m/z 402, m/z 288, and m/z 358 identifies a fragment of P1 peptide:
wherein the m/z 660 identifies a P1 fragment consisting of amino acids EENLR,
the m/z 231 identifies a P1 fragment consisting of amino acids TE,
the m/z 531 identifies a P1 fragment consisting of amino acids ENLR,
the m/z 402 identifies a P1 fragment consisting of amino acids NLR,
the m/z 288 identifies a P1 fragment consisting of amino acids LR, and
the m/z 358 identifies a P1 fragment consisting of amino acids LR doubly charged; and
wherein when the quantified amount of P1 peptide is proportionate to the amount of the p53 isoform in the sample or portion of the sample, and wherein when the p53 isoform in the sample is higher than a control, the presence in the subject of Alzheimer's disease or prognosis of cognitive decline leading to dementia is thereby indicated.

In another aspect, the invention provides a method to provide a composition of one or more fragments of P1 peptide (TEEENLR, SEQ ID NO: 1) comprising one or more mass to charge ratio(s) (m/z) selected from the group consisting of 660, 531, 402, 358, 288, and 231, the method comprising the steps of:
contacting a biological sample of a test subject with an anti-p53 isoform antibody comprising heavy chain CDR sequences (CDR1 (SEQ ID NO:8), CDR2 (SEQ ID NO: 9) and CDR3 (SEQ ID NO: 10)) and light chain CDR sequences (CDR1 (SEQ ID NO:11), CDR2 (SEQ ID NO: 12) and CDR3 (SEQ ID NO: 13)) in a reaction mixture, wherein the presence of p53 isoform in the sample permits formation of an immunocomplex comprising the p53 isoform and the antibody, wherein the p53 isoform is conformationally altered with respect to the wild-type p53 protein;
eluting the p53 isoform to provide eluted p53 isoform;
subjecting the eluted p53 isoform or a portion thereof to enzymatic digestion, thereby to generate one or more proteolytic peptides comprising a P1 peptide (TEEENLR, SEQ ID NO: 1);
performing mass spectrometry on the P1 peptide thereby generating the one or more fragments of P1 peptide,
wherein a mass to charge ratio selected from the group consisting of: m/z 660, m/z 231, m/z 531, m/z 402, m/z 288, and m/z 358 identifies a fragment of P1 peptide:
wherein the m/z 660 identifies a P1 fragment consisting of amino acids EENLR,
the m/z 231 identifies a P1 fragment consisting of amino acids TE,
the m/z 531 identifies a P1 fragment consisting of amino acids ENLR,
the m/z 402 identifies a P1 fragment consisting of amino acids NLR,
the m/z 288 identifies a P1 fragment consisting of amino acids LR, and
the m/z 358 identifies a P1 fragment consisting of amino acids LR doubly charged,
and wherein the composition of one or more fragments of P1 peptide optionally comprises a mass to charge ratio (m/z) of 445 that identifies P1 peptide.

In another aspect, the invention provides a method of generating a mass spectrum indicating the presence of P1 peptide (TEEENLR (SEQ ID NO: 1)), the method comprising the steps of:
providing a biological sample of a subject
contacting the biological sample or a portion thereof with an anti p53 isoform antibody in a reaction mixture, the antibody comprising heavy chain CDR sequences (CDR1 (SEQ ID NO:8), CDR2 (SEQ ID NO: 9) and CDR3 (SEQ ID NO: 10)) and light chain CDR sequences (CDR1 (SEQ ID NO:11), CDR2 (SEQ ID NO: 12) and CDR3 (SEQ ID NO: 13)) to form an immunocomplex comprising the p53 isoform and the antibody the isoform being conformationally altered with respect to the wild-type p53 protein;
eluting the p53 isoform to provide eluted p53 isoform;
subjecting the eluted p53 isoform or a portion thereof to enzymatic digestion to generate a composition comprising one or more proteolytic peptides comprising P1 peptide;
subjecting the P1 peptide of the composition to mass spectrometry to thereby generate a mass spectrum,
wherein the mass spectrum comprises at least one mass to charge ratio selected from the group consisting of: 660, m/z 231, m/z 531, m/z 402, m/z 288, and m/z 358, the group optionally including m/z 445;
wherein the m/z 660 identifies a P1 fragment consisting of amino acids EENLR,
the m/z 231 identifies a P1 fragment consisting of amino acids TE,
the m/z 531 identifies a P1 fragment consisting of amino acids ENLR,
the m/z 402 identifies a P1 fragment consisting of amino acids NLR,
the m/z 288 identifies a P1 fragment consisting of amino acids LR, and
the m/z 358 identifies a P1 fragment consisting of amino acids LR doubly charged,
wherein the mass spectrum comprising the at least one mass to charge ratio selected from the group indicates the presence of the P1 peptide fragment in the composition, and wherein the mass spectrum optionally comprises the m/z 445 that identifies P1 peptide consisting of amino acids TEEENLR.

In some embodiments of any of the above methods, wherein the method further comprises a step of quantifying an amount of the P1 peptide, wherein the quantifying comprises determining the intensity of the mass/charge ratio corresponding to at least one fragment of the P1 peptide to provide the quantified amount of P1 peptide, wherein the quantified amount is higher than a control peptide.

In an embodiment of any of the methods, the subject is human.

In some embodiments of any of the above methods, wherein the mass spectrometry is performed in an ion trap mass spectrometer and the at least one P1 peptide fragment comprises a fragment having m/z of 660.

In some embodiments of any of the above methods, wherein the mass spectrometry is performed in an ion trap mass spectrometer and the at least one P1 peptide fragment comprises a fragment having m/z of 531.

In some embodiments of any of the above methods, wherein the mass spectrometry is performed in an ion trap mass spectrometer and the at least one P1 peptide fragment comprises a fragment having m/z of 402.

In some embodiments of any of the above methods, wherein the mass spectrometry is performed in an ion trap mass spectrometer and the at least one P1 peptide fragment comprises a fragment having m/z of 231.

In some embodiments of any of the above methods, wherein the mass spectrometry is performed in an ion trap mass spectrometer or a triple quad mass spectrometer and the P1 peptide fragment comprises a fragment having m/z of 660 and a fragment having m/z of 231.

In some embodiments of any of the above methods, wherein the control peptide is a labeled control peptide.

In some embodiments of any of the above methods, the labeled control peptide is a labeled P1 peptide and is preferably internal to the composition and is represented as a value comprising its concentration in the reaction mixture.

In some embodiments of any of the above methods, wherein the biological sample is subjected to protein plasma depletion prior to the contacting.

In some embodiments of any of the above methods, wherein the protein plasma depletion is accomplished by one or more of: HPLC, a chromatographic column, and/or chemical treatment of the biological sample.

In some embodiments of any of the above methods, wherein the subject is an asymptomatic subject or exhibits mild cognitive impairment.

In some embodiments of any of the above methods, wherein the mass spectrometry is performed in an ion trap mass spectrometer or a triple quad mass spectrometer and the at least one P1 peptide fragment comprises two fragments having m/z of 660 and m/z of 231.

In some embodiments of any of the above methods, wherein the biological sample is blood, plasma, serum, saliva, urine, neuronal cells, or blood cells.

In some embodiments of any of the above methods, wherein the mass spectrometry is performed in a mass spectrometer and the mass spectrometer is one of MALDI-MS, Ion trap mass spectrometer, Linear ion trap mass spectrometer, Kingdon trap mass spectrometer, Paul trap mass spectrometer, Orbitrap mass spectrometer, Quadrupole ion trap mass spectrometer, Triple quadrupole ion trap mass spectrometer, and Ion cyclotron residence trap mass spectrometer.

In some embodiments of any of the above methods, wherein the antibody is a monoclonal antibody.

In some embodiments of any of the above methods, wherein the mass spectrometry is performed in a triple quad mass spectrometer and the at least one P1 peptide fragment has an m/z of 660.

In some embodiments of any of the above methods, wherein the mass spectrometry is performed in a triple quad mass spectrometer and the at least one P1 peptide fragment comprises has an m/z of 531.

In some embodiments of any of the above methods, wherein the mass spectrometry is performed in a triple quad mass spectrometer and the at least one P1 peptide fragment comprises a fragment having an m/z of 402.

In some embodiments of any of the above methods, wherein the mass spectrometry is performed in an ion trap mass spectrometer or a triple quad mass spectrometer and the at least one P1 peptide fragment comprises a fragment having an m/z of 288.

In some embodiments of any of the above methods, wherein the mass spectrometry is performed in a triple quad mass spectrometer and the at least one P1 peptide fragment comprises a fragment having an m/z of 231.

In some embodiments of any of the above methods, wherein the mass spectrometry is performed in an ion trap mass spectrometer or a triple quad mass spectrometer and the P1 peptide fragment comprises a fragment having an m/z of 231 and a fragment having an m/z of 445.

In some embodiments of any of the above methods, wherein a higher amount of the P1 peptide relative to a control indicates Alzheimer's disease or cognitive decline leading to dementia in the subject.

In some embodiments of any of the above methods, wherein the higher amount of the P1 peptide is greater than 0.071 femtomoles per microliter.

In some embodiments of any of the above methods, wherein the method further comprises the step of treating Alzheimer's disease or cognitive decline leading to dementia using chemical or biological drugs.

In some embodiments of any of the above methods, wherein the chemical drugs are selected from the group consisting of selective serotonin reuptake inhibitor, donepezil, galantamine, rivastigmine, suvorexant, brexpiprazole and memantine.

In some embodiments of any of the above methods, wherein the biological drugs are selected from the group consisting of Aducanumab and Lecanemab.

In some embodiments of any of the above methods, wherein the quantifying further comprises determining the intensity of the mass/charge ratio (m/z) corresponding to the P1 peptide and combining it with the intensity of m/z corresponding to the at least one fragment.

In some embodiments of any of the above methods, wherein the quantifying further comprises determining the intensity of m/z corresponding to the P1 peptide and combining it with the intensity of mass/charge ratio corresponding to at least two fragments.

In some embodiments of any of the above methods, wherein the quantifying further comprises determining the intensity of m/z corresponding to the P1 peptide and combining it with the intensity of mass/charge ratio corresponding to at least three fragments.

In some embodiments of any of the above methods, wherein the quantifying further comprises determining the intensity of m/z corresponding to the P1 peptide and combining it with the intensity of mass/charge ratio corresponding to at least four fragments.

In some embodiments of any of the above methods, wherein the quantifying comprises determining the intensity of m/z corresponding to at least two fragments of the P1 peptide having m/z selected from the group consisting of 660, 231, 531, 402, and 288.

### DETAILED DESCRIPTION

The sequence TEEENLR is present in the linear sequence of p53 (that is, wild type p53), however, this sequence is largely inaccessible (or poorly exposed) in the p53 wild type conformation. In contrast, in the specific unfolded conformation U-p53, the sequence TEEENLR is exposed, and proteolysis of isolated U-p53 generates TEEENLR as a proteolytic peptide (P1), which permits detection of P1 as a marker indicating the presence of U-p53. The presence of U-p53 in a subject sample permits diagnosis of a neurodegenerative disease such as Alzheimer's disease, as documented in PCT/IB2019/051785, which discloses a method of identifying and quantifying p53 peptides in a subject plasma sample using mass spectrometry analysis.

The invention relates to identifying one or more fragments of P1 peptide (TEEENLR), which fragments are generated during quantitation of P1 peptide using mass spectrometry. The mass spectrometry generated P1 peptide fragments are identified and quantitated according to the invention.

The presence of one or more fragments of P1 peptide is useful to serve as one or more biomarkers of a neurodegenerative disease, such as Alzheimer's disease (AD).

The identification and/or quantitation of one or more fragments of P1 peptide permits diagnosis of a neurodegenerative disease, such as Alzheimer's disease (AD), Alzheimer's dementia and mild cognitive impairment (MCI).

Identification of the presence of or identification and quantitation of one or more P1 peptide fragments, including or not including unfragmented P1 peptide itself, are within the scope of the invention. That is, mass spectrometry analysis of the presence of P1 peptide in a sample results in fragmentation of P1 peptide and that fragmentation may result in a mixture of unfragmented P1 peptide and its fragments, or a mixture of fragments of P1 peptide in which the P1 peptide is completely fragmented.

The method is based on the identification of one or more fragments of P1 peptide generated using mass spectrometry to diagnose Alzheimer's disease (AD), a predisposition to the development of AD or to diagnose different forms of dementia.

In particular, first, unfolded p53 protein (U-p53) is captured from a subject sample by immunoprecipitation using a specific p53 antibody. The subject may be suspected of being afflicted with AD, for example, at a pre-clinical or prodromal clinical stage of Alzheimer's disease, and cognitive unimpaired subjects (CU). Then, the captured p53 isoform is separated from the antibody, and the separated p53 protein is subjected to proteolytic digestion to generate one or more peptides that include at least the P1 peptide. The reaction mixture containing the P1 peptide is then subjected to liquid chromatography mass spectrometry and fragments of P1 peptide thus generated are detected.

The amount of unfolded p53 protein present in the bio sample from the subject can be quantified by measuring the amount of one or more fragments of P1 peptide and P1 peptide itself. The amount of one or more of fragments of P1 peptide and unfragmented P1 peptide is compared with that of an internal standard which allows one to determine the exact amount of p53 isoform protein (unfolded p53) present in the reaction mixture.

Preferably the internal standard is a labeled P1 peptide which is spiked into the reaction mixture at known amounts. The signal generated by the P1 peptide, and one or more fragments can be compared to that of a standard curve to determine the concentration or amount of the P1 peptide and one or more of its fragments. The amount or concentration of the P1 peptide in turn provides information on the amount or concentration of unfolded p53 protein found in the biological sample of the subject.

By comparing the amount of P1 peptide and one or more fragments of P1 peptide in the test sample with that of the control sample spiked with labeled P1 peptide, one can determine the amount unfolded p53 protein in the test subject, then by comparing the amount of unfolded p53 protein with the amount of unfolded p53 protein found in the dementia free control subjects, one can determine whether the subject has Alzheimer's disease or is experiencing a cognitive decline leading to dementia. Thus, by detecting and quantifying one or more peptide fragments indicating the presence of unfolded p53 protein, valuable information is provided regarding the disease state. The fragment is generated during ionization of the P1 peptide using mass spectrometry. The fragment thus obtained from the P1 peptide is in a molar equivalent to the unfolded p53 protein and has a well-defined retention time and has a characteristic MS-MS spectrum which can be optimized to deliver attomole detection limits.

The method is advantageously fast, requires a small volume of biological sample and it reliably quantifies the amount of U-p53 protein using the amount of P1 fragments generated in each sample being analyzed. The method is surprisingly accurate as the amount of U-p53 protein is determined based on not only the concentration of the P1 peptide but also the concentration of one or more fragments of P1 peptide.

### BRIEF DESCRIPTION OF FIGURES

FIGS. 1a and 1b shows a schematic of various fragmentation patterns for P1 peptide (TEEENLR).

FIG. 2 shows the mass spectra of an exemplary sample showing two major fragments having m/z values of 660 and 231.

FIG. 3 shows the full scan analysis mass spectra of an exemplary sample showing the presence of various fragments of P1 peptide.

FIG. 4 shows a table listing fragments of P1 peptide from an exemplary sample along with mass and charge values.

FIG. 5 shows a high-level process flow chart of how the samples are processed and quantitation is performed.

### Definitions

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

For example, when separating items in a list, “or” or “and/or” shall be interpreted as being inclusive, i.e., the inclusion of at least one, but also including more than one of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

As used herein in the specification and in the claims, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

Although the above discussion discloses various exemplary embodiments of the invention, it should be apparent that those skilled in the art can make various modifications that will achieve some of the advantages of the invention without departing from the true scope of the invention. Any references to the “invention” are intended to refer to exemplary embodiments of the invention and should not be construed to refer to all embodiments of the invention unless the context otherwise requires. The described embodiments are to be considered in all respects only as illustrative and not restrictive.

“Assaying” as used herein denotes testing for or detecting the presence of a substance or material, such as but not limited to, a chemical, an organic compound, an inorganic compound, a metabolic product, a drug, or a drug metabolite, an organism, or a metabolite of such an organism, a nucleic acid, a protein, or a combination thereof. Optionally, assaying denotes measuring the amount of the substance or material. Assaying further denotes an immunological test, a chemical test, an enzymatic test, and the like.

“Analyte” as used herein refer to molecules that can be detected using the devices and methods of the invention. In some embodiments, the analyte is a molecule that can be suspended or dissolved in a liquid. These include molecules such as nucleic acids, amino acids, lipids, saccharides, hormones, proteins, drugs, drugs of abuse, biological warfare agents, toxins, vitamins, steroids, pesticides, industrial chemicals, analogs, derivatives, and metabolites thereof. Preferably, the analyte is unfolded p53 protein (U-p53) in a blood sample.

“Sample” as used herein refers to a biological sample in liquid form, such as blood, urine, plasma, serum, cerebrospinal fluid, saliva. A liquid sample is a biological fluid of a subject, for example, blood, urine, plasma, serum, cerebrospinal fluid, saliva.

“Blood” as used herein refers to fluid that circulates in the heart, arteries, capillaries, and veins of a vertebrate animal carrying nourishment. The term blood also encompasses derivatives of blood such as serum and plasma.

A variety of assays can be used to detect the presence, absence, or concentration of unfolded p53 protein in a sample. For example, unfolded p53 protein (U-p53) can be detected using binding moieties such as antibodies which can be labeled as well, chemicals that react with the unfolded p53 protein, UV based detection of complex formed by the binding of unfolded p53 protein with binding moieties, intact/nature electrospray mass spectrometry, electrochemical detection, or any combination thereof. Antibodies that detect unfolded p53 protein are described in PCT/EP2015/072094 which is incorporated by reference in its entirety. An exemplary antibody that binds to unfolded p53 comprises heavy chain CDRs 1, 2 and 3 (SEQ ID NOS: 8, 9 and 10, respectively) and light chain CDRs 1, 2 and 3 (SEQ ID NOS: 11, 12 and 13, respectively).

An exemplary antibody that binds to unfolded p53 comprises the heavy chain variable region (SEQ ID NO: 6) and light chain variable region (SEQ ID NO: 7),

An exemplary antibody that binds to unfolded p53 comprises the heavy chain (SEQ ID NO: 4) and light chain (SEQ ID NO: 5)

An exemplary antibody that binds to unfolded p53 is 2D3A8, which comprises the heavy chain (SEQ ID NO: 4) and light chain (SEQ ID NO: 5), heavy chain variable region (SEQ ID NO: 6) and light chain variable region (SEQ ID NO: 7), heavy chain CDRs 1, 2 and 3 (SEQ ID NOS: 8, 9 and 10, respectively) and light chain CDRs 1, 2 and 3 (SEQ ID NOS: 11, 12 and 13, respectively).

As used herein, the terms “unfolded” or “conformational variant of” or “misfolded” or “unfolded/misfolded” are used interchangeably. A protein is considered to be misfolded if it cannot achieve its native state of folding. Misfolding can be due to mutations in the amino acid sequence or a disruption of the normal folding process by external factors. A protein is considered to be unfolded (misfolded) when its native conformation is changed because of exposure to one or more of chemical, biological, thermal or mechanical denaturation processes. Unfolded p53 and misfolded p53, are equivalent terms as used herein and refer to an unfolded isoform of the multifunctional protein p53 in plasma which has been known to be present in higher amounts in Alzheimer's Disease subjects in comparison with healthy subjects. U-p53 is an example of the unfolded/misfolded isoform of p53 protein which is bound by anti-p53 antibody such as 2D3A8. See Abate, G., Frisoni, G. B., Bourdon, J C. et al. The pleiotropic role of p53 in functional/dysfunctional neurons: focus on pathogenesis and diagnosis ofAlzheimer's disease. Alz Res Therapy 12, 160 (2020) and Unfolded p53: A Potential Biomarker for Alzheimer's Disease, Lanni et al., Journal of Alzheimer's Disease, vol. 12, no. 1, pp. 93-99, 2007.

A biological sample (blood, plasma, or serum) from a “cognitive normal” subject has less than 0.071 femtomoles/microliter of unfolded p53 protein (U-p53) and this amount is considered as a normal or wild type level of U-p53. A biological sample (blood, plasma, or serum) from a subject afflicted with a neurogenerative disease such as dementia or Alzheimer's disease has greater than 0.071 femtomoles/microliters of U-p53 protein.

A biological sample from a subject having no symptoms or mild cognitive impairment (MCI) but having 0.071 femtomoles/microliter of unfolded p53 protein (U-p53) has a moderate risk of progressing to Alzheimer's dementia within 5-6 years.

A biological sample from a subject having no symptoms or mild cognitive impairment (MCI) but having greater than 0.071 femtomoles/microliter of unfolded p53 protein (U-p53) has a high risk of progressing to Alzheimer's dementia within 5-6 years.

A biological sample from a subject having no symptoms or mild cognitive impairment (MCI) but having less than 0.071 femtomoles/microliter of unfolded p53 protein (U-p53) has a low risk of progressing to Alzheimer's dementia within 5-6 years.

As used herein, the term “moderate risk progressor” refers to a subject who has minimal or no symptom of cognitive decline and has 0.071 femtomoles/microliter of unfolded p53 protein (U-p53) detected in the biological sample. This subject has a moderate risk of progressing into AD within five to six years of testing.

As used herein, the term “high risk progressor” refers to a subject who has minimal or no symptom of cognitive decline and has greater than 0.071 femtomoles/microliter of unfolded p53 protein (U-p53) detected in the biological sample. This subject has a high risk of progressing into AD within five to six years of testing.

As used herein, the term “low risk progressor” refers to a subject who has minimal or no symptom of cognitive decline and has less than 0.071 femtomoles/microliter of unfolded p53 protein (U-p53) detected in the biological sample. This subject has a low risk of progressing into AD within five to six years of testing.

“Neurodegenerative disease” refers to a wide range of conditions that result from progressive damage to cells and nervous system connections that are essential for mobility, coordination, strength, sensation, and cognition. Some non-limiting examples of neurodegenerative diseases include Alzheimer's disease, Mild cognitive impairment, Lewy body dementia, Fronto temporal dementia, and Vascular dementia.

“Dementia” as used herein refers a neurological disease state wherein subjects experience symptoms of impairment in memory, communication, and thinking. Possible symptoms of dementia include one or more of cognitive changes such as recent memory loss, difficulty completing familiar and/or routine tasks, problems communicating thoughts to others, disorientation, problems with abstract thinking, misplacing things, and loss of initiative. It can be also accompanied with one or more of psychological changes such as mood changes, personality changes, depression, anxiety, inappropriate behavior, paranoia, agitation, and hallucinations.

“Indicate” (the verb) means to signify the presence of a component, or to signify a certain condition, or to be proportionate to a certain constituent.

“Indicators of dementia” as used herein refers to the following symptoms which are commonly present in subjects with dementia. They include memory loss, hallucination, paranoia, aggressive behavior, and depression. In some subjects, two or more indicators of dementia are present. In some subjects two indicators of dementia are present. In some subjects, three indicators of dementia are present. In some subjects, four indicators of dementia are present.

“Dementia stages” as used herein refers to developmental stages of the disease which becomes progressively debilitating to the subject. Dementia is roughly split into four stages, (a) Mild cognitive impairment (b) Mild dementia, (c) Moderate dementia and (d) Severe dementia.

“Mild Cognitive Impairment (MCI)” as used herein is a very early stage of dementia characterized by general forgetfulness. This affects many people as they age but it only progresses to dementia for some.

“Mild dementia” as used herein refers to a stage of dementia wherein subject with mild dementia will experience cognitive impairments that occasionally impact their daily life. Symptoms include memory loss, confusion, personality changes, getting lost, and difficulty in planning and carrying out tasks.

“Moderate dementia” as used herein refers to a stage of dementia wherein subject's daily life becomes more challenging, and the individual may need more help. Symptoms are similar to mild dementia but increased. Individuals may need help getting dressed and combing their hair. They may also show significant changes in personality; for instance, becoming suspicious or agitated for no reason. There are also likely to be sleep disturbances.

“Severe dementia” as used herein refers to an advanced stage of dementia wherein the subject's symptoms have worsened considerably. There may be a loss of ability to communicate, and the individual might need full-time care. Simple tasks, such as sitting and holding one's head up become impossible. Bladder control may be lost.

“Progressive dementias” as used herein refers to dementias that progress and are not reversible in nature, but the progression can be slowed down but not prevented. Common examples of progressive dementias include Alzheimer's disease, Vascular dementia, Lewy body dementia, Frontotemporal dementia, and Mixed dementia.

“Alzheimer's disease” as used herein refers to neurological disorder which is believed to be the most common cause of dementia. Alzheimer's disease subject has plaques and tangles in their brains. Plaques are clumps of a protein called beta-amyloid, and tangles are fibrous tangles made up of tau protein. It is thought that these clumps damage healthy neurons and the fibers connecting them.

“Vascular dementia” as used herein refers to the most common type of dementia is caused by damage to the vessels that supply blood to your brain. Blood vessel problems can cause strokes or damage the brain in other ways, such as by damaging the fibers in the white matter of the brain. The most common symptoms of vascular dementia include difficulties with problem-solving, slowed thinking, focus, and organization. These tend to be more noticeable than memory loss.

“Lewy body dementia” as used herein refers to dementia found in subjects with Lewy bodies. Lewy bodies are abnormal balloon like clumps of protein that have been found in the brain of a subject with Lewy body dementia, Alzheimer's disease and Parkinson's disease. Common symptoms include acting out one's dreams in sleep, visual hallucinations, and problems with focus and attention. Other symptoms include uncoordinated or slow movement, tremors, and rigidity (parkinsonism).

“Frontotemporal dementia” as used herein refers to a group of diseases characterized by the breakdown (degeneration) of nerve cells and their connections in the frontal and temporal lobes of the brain, the areas generally associated with personality, behavior, and language. Common symptoms affect behavior, personality, thinking, judgment, and language and movement.

“Mixed dementia” as used herein refers to dementia that is caused by multiple neurological disorders such as Alzheimer's disease, vascular dementia, and Lewy body dementia. Elderly subjects over the age of eighty are prone to get mixed dementia.

“Dementia linked disorders” as used herein refers to neurological disorders where subjects having one or more of Huntington's disease, Traumatic brain injury (TBI). Creutzfeldt-Jakob disease and Parkinson's disease exhibit symptoms of dementia.

“Prognosis” as used herein, refers to forecasting of the probable course and outcome of a disease, especially of the chances of recovery. It indicates the prospect of recovery as anticipated from the course of disease.

“Predicting the onset of dementia” as used herein refers to predicting the expected development of dementia, including whether the symptoms of dementia will improve or worsen or remain stable over time. Such a prediction carries with it the expectations of quality of life, such as the ability to carry out daily activities; the potential for complications and associated health issues; and the likelihood of survival (including life expectancy). Typically, a subject seeking prognosis for the onset of dementia have not yet exhibited any of the common symptoms of dementia. Subject seeking prognosis may have one or more close relatives who suffered or suffer from dementia, and therefore might be hereditarily prone to dementia. For instance, prediction of the onset involves determining whether subject having no symptoms of dementia will likely develop dementia in a follow up period up 10 years by analyzing a subject's blood sample for the presence of unfolded p53 protein U-p53.

“Diagnosis” as used herein, refers to the process of determining by examination the nature and circumstances of a diseased condition.

“Detecting or confirming the presence of dementia” as used herein refers to analyzing a liquid biological sample of a subject having one or more symptoms of dementia for the presence of an analyte such as unfolded p53 protein.

“Onset” as used herein refers to the beginning or commencement.

“Onset of dementia” in a subject refers to when a subject first exhibits and/or experiences one or more symptoms of dementia.

“Predict” refers to the ability to foretell on the basis of observation, experience, or scientific reason the occurrence of an event.

“Management of dementia” as used herein refers to one or more actions that the subject diagnosed with dementia or predicted to get dementia in the next 5-10 years can take to slowdown the progression and/or reduce the severity of dementia. These actions include but not limited to (a) Mentally stimulating activities, such as reading, solving puzzles and playing word games, (b) Being physically and socially active, (c) Avoid smoking, (d) Treat high blood pressure, high cholesterol, diabetes, and lower high body mass index (BMI), (e) Increase quality of sleep and (f) Maintain healthy diet and reduce vitamin D deficiency.

“Reversible dementia” as used herein refers to dementia or dementia-like symptoms can be reversed with treatment. Reversible dementia occurs due to one or more of infections and immune disorders, Metabolic problems, and endocrine abnormalities, Nutritional deficiencies, Medication side effects, Subdural hematomas, Brain tumors, and Anoxia. Addressing these causes would enable the eradication of dementia symptoms.

“Risk factors for dementia” as used herein refers to one or more factors that are believed to be associated with increasing the chances of getting dementia, these include heavy alcohol use, elevated cardiovascular risk due to high body mass index (BMI), depression, diabetes, smoking, sleep apnea, and vitamin deficiencies involving low levels of vitamin D, vitamin B-6, vitamin B-12 and folate.

“Symptoms of dementia” as used herein refers to one or more cognitive and/or psychological changes that signal the presence or onset of dementia. Examples of cognitive change include one or more of memory loss, difficulty communicating, difficulty with visual and spatial abilities, difficulty with reasoning problem-solving, difficulty handling complex tasks, difficulty with planning and organizing, difficulty with coordination and motor functions, and confusion and disorientation. Examples of psychological change include one or more of depression, anxiety, paranoia, agitation, and hallucinations.

“Genetic predisposition to dementia” as used herein refers to a condition wherein the subject has close family relatives such as siblings, parents, or grandparents who have been diagnosed with dementia. It also includes subject who has abnormal expression of or have mutations that affect function of one or more genes that are believed to play a role in the onset of dementia. These dementia related genes are selected from the group consisting of APOE4, ABCA7, CLU, CR1, PICALM, PLD3, TREM2, SORL1, APP, PSEN1 and PSEN2.

“Cognitive Decline” as used herein refers to a condition wherein the individual shows a reduced ability to learn new information, a higher ability to become distracted, and a slower mental process, memory loss.

“Subjective memory complaints” as used herein refers to a condition wherein an individual experiences self-reported problems with memory that may or may not present with objective cognitive impairment (measured via tests and assessments).

As used herein, the term “P1 fragment” or “fragment of P1 peptide” refers to charged ions of P1 peptide generated by the mass spectrometer. The fragments comprise one or more amino acid residues. Generally, the fragments of P1 peptide range from one amino acid residue in length to six amino acids in length. A subset of fragments of P1 peptide are shown below as a non-limiting example:

P1 Fragment Sequence

TEEEN
TEEE
TEE
TE
EENLR
ENLR
NLR
LR
R
TEEENL

The fragmentation pattern of P1 peptide being generated is dependent on factors like capillary voltage, collision gas pressure and temperature along with the type of mass spectrometer such as ion trap or triple quadrupole etc. Exemplary embodiments of P1 fragments are shown in SEQ ID Nos 15-23.

As used herein, the term “Collision-induced dissociation (CID)” refers to a technique used in mass spectrometry to induce fragmentation of selected ions in the gas phase. The selected ions (typically molecular ions or protonated molecules) are usually accelerated by applying an electrical potential to increase the ion kinetic energy and then allowed to collide with neutral molecules (often helium, nitrogen, or argon).

In an ion trap (Paul trap) mass spectrometer, Orbitrap (modified Kingdon trap) or an ion cyclotron resonance mass spectrometer (aka FT-MS), the ions are trapped in a static electric or magnetic field, and the collision gas is introduced into the trap. The ions are then allowed to collide with the collision gas, which can induce fragmentation. The mass spectrometer then scans the fragment ions, recording their mass-to-charge ratios (m/z).

This difference in how the collision gas is introduced can lead to some differences in the fragmentation patterns observed. In general, the fragmentation patterns observed in an ion trap are more complex than those observed in a triple quadrupole mass spectrometer. This is because the ions in an ion trap have more time to interact with the collision gas, which can lead to more extensive fragmentation.

As used herein, the term “MALDI-MS” refers to matrix-assisted laser desorption/ionization (MALDI) which is an ionization technique that uses a laser energy-absorbing matrix to create ions from large molecules with minimal fragmentation. It has been applied to the analysis of biomolecules (biopolymers such as DNA, proteins, peptides, and carbohydrates) and various organic molecules (such as polymers, dendrimers, and other macromolecules), which tend to be fragile and fragment when ionized by more conventional ionization methods. It is similar in character to electrospray ionization (ESI) in that both techniques are relatively soft (low fragmentation) ways of obtaining ions of large molecules in the gas phase, though MALDI typically produces far fewer multi-charged ions. (See Hillenkamp F, Karas M, Beavis R C, Chait B T. Matrix-assisted laser desorption/ionization mass spectrometry of biopolymers. Anal Chem. 1991 Dec. 15; 63(24):1193A-1203A.). MALDI methodology is a three-step process. First, the sample is mixed with a suitable matrix material and applied to a metal plate. Second, a pulsed laser irradiates the sample, triggering ablation and desorption of the sample and matrix material. Finally, the analyte molecules are ionized by being protonated or deprotonated in the hot plume of ablated gases, and then they can be accelerated into whichever mass spectrometer is used to analyze them.

As used herein, the term “Ion trap mass spectrometer” refers to a mass spectrometer that uses ion trap to capture charged particles. An ion trap is a combination of electric and/or magnetic fields used to capture charged particles known as ions often in a system isolated from an external environment. An ion trap mass spectrometer may incorporate a Penning trap (Fourier-transform ion cyclotron resonance) (Blaum, Klaus (2006). “High-accuracy mass spectrometry with stored ions”. Physics Reports. 425 (1): 1-78.), Paul trap (Douglas, D J; Frank, A J; Mao, D M (2005). “Linear ion traps in mass spectrometry”. Mass Spectrometry Reviews. 24 (1): 1-29.) or the Kingdon trap. (Kingdon K H (1923). “A Method for the Neutralization of Electron Space Charge by Positive Ionization at Very Low Gas Pressures”. Physical Review. 21 (4): 408-418.), The Orbitrap, introduced in 2005, is based on the Kingdon trap. (The Orbitrap: a new mass spectrometer”. Journal of Mass Spectrometry. 40 (4): 430-443). Other types of mass spectrometers may also use a linear quadrupole ion trap as a selective mass filter.

As used herein, the term “Linear ion trap” or LIT is a type of ion trap mass spectrometer. In a LIT, ions are confined radially by a two-dimensional radio frequency (RF) field, and axially by stopping potentials applied to end electrodes. LITs have high injection efficiencies and high ion storage capacities. The LIT uses a set of quadrupole rods to confine ions radially and a static electrical potential on the end electrodes to confine the ions axially. (“Linear ion traps in mass spectrometry”. Mass Spectrometry Reviews. 24 (1): 1-29). The LIT can be used as a mass filter or as a trap by creating a potential well for the ions along the axis of the trap. (Quadrupole; March, Raymond E.; Spectrometry, Mass (2000). “Quadrupole ion trap mass spectrometry: a view at the turn of the century”. International Journal of Mass Spectrometry. 2000 (1-3): 285-312). The mass of trapped ions may be determined if the m/z lies between defined parameters. (Peng, Ying; Austin, Daniel E. (November 2011). “New approaches to miniaturizing ion trap mass analyzers”. TrAC Trends in Analytical Chemistry. 30 (10): 1560-1567). Advantages of the LIT design are high ion storage capacity, high scan rate, and simplicity of construction.

As used herein, the term “Kingdon trap” is a type of ion trap mass spectrometer. A Kingdon trap consists of a thin central wire, an outer cylindrical electrode and isolated end cap electrodes at both ends. A static applied voltage results in a radial logarithmic potential between the electrodes. (A Method for the Neutralization of Electron Space Charge by Positive Ionization at Very Low Gas Pressures”. Physical Review. 21 (4): 408-418.). In a Kingdon trap there is no potential minimum to store the ions; however, they are stored with a finite angular momentum about the central wire and the applied electric field in the device allows for the stability of the ion trajectories.

As used herein, the term “Paul trap” is another type of ion trap that uses static direct current (DC) and radio frequency (RF) oscillating electric fields to trap ions.

As used herein, the term “Orbitrap” is a type of ion trap mass analyzer that consists of two outer electrodes and a central electrode, which enable it to act as both an analyzer and detector. It consists of an outer barrel-like electrode and a coaxial inner spindle-like electrode that traps ions in an orbital motion around the spindle. (Makarov, A (2000). “Electrostatic axially harmonic orbital trapping: A high-performance technique of mass analysis”. Analytical Chemistry. 72 (6): 1156-62.) The image current from the trapped ions is detected and converted to a mass spectrum using the Fourier transform of the frequency signal. (Hu, Q; Noll, R J; Li, H; Makarov, A; Hardman, M; Graham Cooks, R (2005). “The Orbitrap: a new mass spectrometer”. Journal of Mass Spectrometry. 40 (4): 430-43) As used herein the term “Quadrupole ion trap” is a type of mass analyzer that consists of three hyperbolic electrodes: a donut-shaped ring electrode, an entrance endcap electrode, and the exit endcap electrode. These electrodes form a cavity in which it is possible to trap (store) and analyze the ions. Both endcap electrodes have a hole in their center through which the ions can travel, and the ring electrode is located midway between the two endcap electrodes. Ions enter the quadrupole ion trap through the entrance endcap electrode. The ions are then trapped in the space between the electrodes by AC (oscillating, nonstatic) and DC (nonoscillating, static) electric fields. Various voltages are applied to the electrodes to trap and eject ions based on their m/z. The ring electrode RF potential produces a 3D quadrupolar potential field within the trapping cavity, which traps ions in a stable oscillating trajectory that is confined within the trapping cell. The exact nature of the trajectory depends on the trapping potential and the m/z value of the ions. Then, the electrode system potentials are altered to produce instabilities in the ion trajectories, causing the ions to be axially ejected in order of increasing m/z value and focused by the exit lens prior to detection. An important distinction between quadrupole mass analyzers and quadrupole ion trap mass analyzers is that quadrupole mass analyzers separate and detect masses by allowing oscillating ions to pass through the quadrupole to reach the detector (beam-type mass analyzer), whereas quadrupole ion trap mass analyzers separate and detect masses by discharging ions with unstable oscillations from the system (trapping-type mass analyzer).

As used herein, the term “Triple quadrupole ion trap” refers to a triple quadrupole mass spectrometer, also known as simply a triple quad, is a type of mass spectrometer commonly used in analytical chemistry and biochemistry. It is a highly sensitive instrument that is capable of precise and selective analysis of complex mixtures of chemical compounds.

The triple quadrupole mass spectrometer consists of three quadrupole mass analyzers arranged in series. Each quadrupole consists of four parallel rods that create an electric field. These electric fields allow the instrument to selectively filter and manipulate ions based on their mass-to-charge ratio (m/z).

The first quadrupole, often referred to as the “mass filter” or “Q1,” acts as a mass-selective filter and allows only ions of a specific m/z ratio to pass through. This quadrupole can be used to isolate and filter out ions of interest from a complex sample.

The second quadrupole, known as the “collision cell” or “Q2,” is where targeted ions from Q1 are subjected to collision-induced dissociation (CID) or other fragmentation techniques. This process breaks down the ions into smaller fragments, which can provide information about the structure and composition of the original molecules.

The third quadrupole, called the “mass analyzer” or “Q3,” acts as a mass filter again, but this time it is used to selectively detect and measure specific fragments generated in Q2. By scanning the voltage applied to Q3, one can measure the abundance of specific fragments and generate a mass spectrum.

The triple quadrupole mass spectrometer is often used in tandem mass spectrometry (MS/MS) experiments, where the instrument can perform precursor ion selection in Q1, fragmentation in Q2, and detection of specific fragments in Q3. This allows for highly specific and sensitive analysis of targeted compounds in complex samples.

Overall, the triple quadrupole mass spectrometer offers high sensitivity, selectivity, and quantification capabilities, making it a valuable tool in various fields such as pharmaceutical analysis, environmental monitoring, metabolomics, and proteomics. As used herein, the term “Ion cyclotron resonance trap” refers to an ion cyclotron resonance trap (ICR trap) is a type of mass spectrometer that utilizes the principles of ion cyclotron resonance (ICR) to analyze and measure the properties of ions. It is a powerful instrument for high-resolution mass spectrometry.

In an ICR trap, ions are trapped and confined within a magnetic field created by superconducting magnets. The magnetic field causes the ions to move in circular paths, forming an ion cyclotron motion. The frequency of the ion's cyclotron motion is directly proportional to its mass-to-charge ratio (m/z).

The ICR trap consists of a cylindrical or toroidal chamber where the ions are trapped. The magnetic field is typically homogeneous in the center of the trap, allowing the ions to move in circular paths with well-defined frequencies. Radiofrequency (RF) voltage is applied to the trapping electrodes, creating an oscillating electric field that interacts with the trapped ions.

To measure the properties of the trapped ions, various techniques can be employed. One common approach is to apply a short burst of additional RF voltage to the trapping electrodes, which causes the ions to oscillate at their characteristic frequencies. The resulting ion motion induces a current in the detection electrodes, which is detected and analyzed.

The time it takes for the ions to complete one cycle of their cyclotron motion can be measured precisely, allowing for accurate determination of their mass-to-charge ratio. The ICR trap can achieve extremely high mass resolution, often exceeding one million, making it a valuable tool for identifying and characterizing complex mixtures of ions.

ICR traps are widely used in various research areas, including proteomics, metabolomics, petroleomics, and environmental analysis. They offer exceptional mass accuracy, resolving power, and the ability to study complex ion structures and dynamics. However, ICR traps require sophisticated instrumentation and are typically operated under high vacuum conditions, making them more complex and expensive compared to other mass spectrometry techniques.

As used herein, the term “Specific setting” refers to the settings under which the mass spectrometry (MS) analysis should be performed. The MS analysis is done with settings that a generically lower than the normal operating needle voltages. These differ from manufacture and source type but they are always lower than the manufacturers default settings. The product ion at m/z of 660 arising from the P1 peptide of m/z of 445 is the most diagnostic fragment. The label 15N Arginine or D10 Leucine both have the transition of m/z 450 to 670.

As used herein, the term “reaction mixture” refers to a liquid mixture of components permitting interaction among the components.

As used herein, the term “m/z” of “mass to charge ratio” refers to the ratio that is expressed in units of mass divided by units of charge, typically in atomic mass units (amu) divided by elementary charge (e). The elementary charge is the charge of a proton, approximately 1.602×10⁻¹⁹ coulombs.

As used herein, the term “peptide” refers to short chains of amino acids linked by peptide bonds. Peptides are molecules that consist of between 2 and 30 amino acids. Peptides are different from proteins which are much larger with greater than 50 amino acids.

As used herein the term “isolate” or “isolating” refers to the process of separating a component of interest such as a peptide or a protein, by using chemical or physical methods from a mixture of multiple components.

As used herein the term “mass spectrum” refers to a histogram plot or vertical bar graph of intensity vs. mass-to-charge ratio (m/z) in a chemical or biological sample acquired using a mass spectrometer. Each bar represents an ion having a specific mass-to-charge ratio (m/z) and the length of the bar indicates the relative abundance of the ion.

As used herein the term “spectrum” refers to a collection of one or more fragments of peptide having unique m/z ratios that are generated when a sample containing a peptide is subjected to mass spectrometry.

As used herein, the term “Control value” refers to a threshold concentration of U-p53 protein above which a person is diagnosed as having high risk of either having AD or developing AD in the next 5-6 years. The control value is 0.071 femtomoles per microliter of U-p53 protein.

As used herein, the term “Control peptide” refers to a known amount of peptide which serves as control for detection and quantification of signal during an assay such as mass spectrometry. Preferably the control peptide is a P1 peptide (TEEENLR) but it could be other peptides of p53 protein. In some instances, the control peptide is labeled so it can be distinguished from the sample and is referred to as “Labeled control peptide”. In some instances, the control peptide is internal and is added to the sample prior to an assay such as mass spectrometry.

As used herein, the term “Control peptide fragment” refers to byproducts of disintegration of the control peptide during the mass spectrometry assay. Preferably the control peptide fragment is the fragment of P1 peptide which is labeled. Since the amount of control peptide added to a sample is known, one can correlate the control peptide fragment generated from the labeled control peptide with the signal generated in the mass spectrometry. This correlation is then relied upon to determine the amount of peptide fragments present in the sample.

As used herein the term “fmol” refers to femtomoles.

### Treatment of Alzheimer's Disease and Dementia

A subject that is classified as having AD or having cognitive decline leading to dementia progressing to AD by any of the aforesaid methods is then treated by a medical professional according to acceptable medical standards.

A subject determined to be at high risk of AD or not at high risk but at moderate risk of AD and possibly some low-risk subjects may be determined by a skilled medical profession to require a certain treatment regimen. For example, a skilled medical professional may determine that a disease-modifying treatment, disease-modifying drug, or disease-modifying therapy is applicable to a given subject. Such a treatment may delay or slow the progression of a disease by targeting its underlying cause.

In some embodiments, the method is useful in stratifying participants in early AD clinical trials for measuring effectiveness of novel drugs for AD treatment. In some embodiments, the method is useful for triaging subjects in clinical settings on risk-benefit basis for treatment with AD drugs. In some embodiments, the method is useful for individuals to plan for scenarios such as DNR, Will and advanced care treatments.

In some embodiments, the method is useful in recruiting participants in primary prevention studies (e.g. AHEAD, DIAN) who may have the brain pathology of AD (confirmed by amyloid and tau testing) but are not at a high-risk for immediate deterioration. In some embodiments, the method is useful for triaging subjects in clinical settings and to identify individuals who are at risk for developing AD by monitoring the level of P1 peptide and its one or more fragments at regular intervals such as 6-12 months. In some embodiments, the method is useful for individuals who have family history of dementia or AD to test their personal risk profile.

In some embodiments, the method further comprises the step of treating the subject classified as one of high risk, moderate risk, or low risk of progression to AD.

In some embodiments of any of the aforesaid methods, the subject is classified as high risk progressor if the U-p53 protein is present in concentrations greater than 0.071 fmoles/microliter then the subject is classified as a high risk progressor of AD. A high risk progressor is at high risk of progressing into AD within the next 6 years. The subject thus classified as a high risk progressor could be then treated with one or more disease modifying medications comprising one or more chemical and/or biological drugs selected from the group consisting of selective serotonin reuptake inhibitor, donepezil, galantamine, rivastigmine, suvorexant, brexpiprazole, memantine, aducanumab, and lecanemab or combinations thereof.

In some embodiments of any of the aforesaid methods, the subject, 6-12 months post treatment is tested again following the same procedures outlined above to see if the concentration of U-p53 protein is increased or decreased or stayed the same.

In some embodiments of any of the aforesaid methods, if the concentration of U-p53 protein increases post treatment for a high risk progressor, then the dosage and/or frequency of administration is increased as it might imply the drug is not effective at the dosage being administered. If the concentration of U-p53 protein decreases post treatment for a high risk progressor, then the drug being administered is considered to be effective in treating AD and the treatment regimen is maintained. If the concentration of U-p53 protein remains unchanged post treatment for a high risk progressor, then the drug being administered is considered to be effective at preventing the progression of AD and optionally the drug dosage is incrementally increased to determine optimal dose for the subject.

In some embodiments of any of the aforesaid methods, the subject is classified as a slow progressor or a low risk progressor if the U-p53 protein is present in concentrations less than 0.071 fmoles/microliter then the subject is classified as a low risk progressor of AD. A low risk progressor is at low risk of progressing into AD within the next 6 years. The subject thus classified as a low risk progressor is then treated with one or more MIND (Mediterranean-DASH Intervention for Neurodegenerative Delay) diet, DASH (Dietary Approaches to Stop Hypertension) diet, vitamin/antioxidant supplement, blood pressure reducing medication and anti-inflammatory medication.

In some embodiments of any of the aforesaid methods, the subject thus classified as a low risk progressor is then treated with one or more MIND (Mediterranean-DASH Intervention for Neurodegenerative Delay) diet, DASH (Dietary Approaches to Stop Hypertension) diet, vitamin/antioxidant supplement, blood pressure reducing medication and anti-inflammatory medication.

In some embodiments of any of the aforesaid methods, the vitamin/antioxidant supplement is one or more of vitamin A, vitamin B, folic acid, vitamin C, vitamin D, vitamin E, DHA (docosahexaenoic acid), ubiquinone, lycopene, coenzyme Q10 and ellagic acid, ascorbic acid, masoprocol, pramipexole, nitric oxide, allopurinol, pentoxifylline, melatonin, probucol, quercetin, acetylcysteine, n acetylcysteine, acetyl-L-carnitine and 1-methylfolate and resveratrol. (Mielech A, Puścion-Jakubik A, Markiewicz-Zukowska R, Socha K. Vitamins in Alzheimer's Disease-Review of the Latest Reports. Nutrients. 2020 Nov. 11; 12(11):3458).

In some embodiments of any of the aforesaid methods, the blood pressure reducing medication is selected from the group consisting of Angiotensin-converting enzyme (ACE) inhibitors, Angiotensin receptor blockers (ARBs), Calcium-channel blockers and Beta-blockers.

In some embodiments of any of the aforesaid methods, the anti-inflammatory medication is selected from the group consisting of Aspirin, Diclofenac, Etodolac, Fenoprofen, Flurbiprofen, Ibuprofen, Indomethacin, Meclofenamate, Mefenamic Acid, Nabumetone, Naproxen, Oxaprozin, Piroxicam, Sulindac, Tolmetin, Celecoxib, and Meloxicam.

In some embodiments of any of the aforesaid methods, the subject, 6-12 months post treatment is tested again following the same procedures outlined above to see if the concentration of U-p53 protein is increased or decreased or stayed the same.

In some embodiments of any of the aforesaid methods, if the concentration of U-p53protein increases post treatment, then the treatment regimen is not effective and is discontinued. If the concentration of U-p53 protein decreases post treatment, then the treatment regimen being administered is considered to be effective in preventing AD and the treatment regimen is maintained. If the concentration of U-p53 protein remains unchanged post treatment, then the drug being administered is considered to be effective at preventing the progression of AD and optionally the additional lifestyle changes such as physical exercise can be added to the treatment regimen.

All combinations of the preferred aspects of the peptides of the invention, preparation processes, and methods disclosed above are to be understood as herein described. Below are working examples of the present invention provided for illustrative purposes.

### SEQUENCES

```
SEQ ID NO: 1-Linear Epitope
Arg Arg Thr Glu Glu Glu Asn Leu Arg Lys Lys Gly Glu Pro His His
1 5 10 15
SEQ ID NO: 2-Immunization Peptide
Cys Arg Thr Glu Glu Glu Asn Leu Arg Lys Lys Gly Glu Pro His His
1 5 10 15
SEQ ID NO: 3-p53 Protein
Met Glu Glu Pro Gln Ser Asp Pro Ser Val Glu Pro Pro Leu Ser Gln
1 5 10 15
Glu Thr Phe Ser Asp Leu Trp Lys Leu Leu Pro Glu Asn Asn Val Leu
                        20 25 30
Ser Pro Leu Pro Ser Gln Ala Met Asp Asp Leu Met Leu Ser Pro Asp
                35 40 45
Asp Ile Glu Gln Trp Phe Thr Glu Asp Pro Gly Pro Asp Glu Ala Pro
        50 55 60
Arg Met Pro Glu Ala Ala Pro Pro Val Ala Pro Ala Pro Ala Ala Pro
65 70 75 80
Thr Pro Ala Ala Pro Ala Pro Ala Pro Ser Trp Pro Leu Ser Ser Ser
                                85 90 95
Val Pro Ser Gln Lys Thr Tyr Gln Gly Ser Tyr Gly Phe Arg Leu Gly
                        100 105 110
Phe Leu His Ser Gly Thr Ala Lys Ser Val Thr Cys Thr Tyr Ser Pro
                115 120 125
Ala Leu Asn Lys Met Phe Cys Gln Leu Ala Lys Thr Cys Pro Val Gln
        130 135 140
Leu Trp Val Asp Ser Thr Pro Pro Pro Gly Thr Arg Val Arg Ala Ala
145 150 155 160
Ile Tyr Lys Gln Ser Gln His Met Thr Glu Val Val Arg Arg Cys Pro
                                165 170 175
His His Glu Arg Cys Ser Asp Ser Asp Gly Leu Ala Pro Pro Gln His
                        180 185 190
Leu Ile Arg Val Glu Gly Asn Leu Arg Val Glu Tyr Leu Asp Asp Arg
                195 200 205
Asn Thr Phe Arg His Ser Val Val Val Pro Tyr Glu Pro Pro Glu Val
        210 215 220
Gly Ser Asp Cys Thr Thr Ile His Tyr Asn Tyr Met Cys Asn Ser Ser
225 230 235 240
Cys Met Gly Gly Met Asn Arg Arg Pro Ile Leu Thr Ile Ile Thr Leu
                                245 250 255
Glu Asp Ser Ser Gly Asn Leu Leu Gly Arg Asn Ser Phe Glu Val Arg
                        260 265 270
Val Cys Ala Cys Pro Gly Arg Asp Arg Arg Thr Glu Glu Glu Asn Leu
                275 280 285
Arg Lys Lys Gly Glu Pro His His Glu Leu Pro Pro Gly Ser Thr Lys
        290 295 300
Arg Ala Leu Pro Asn Asn Thr Ser Ser Ser Pro Gln Pro Lys Lys Lys
305 310 315 320
Pro Leu Asp Gly Glu Tyr Phe Thr Leu Gln Ile Arg Gly Arg Glu Arg
                                325 330 335
Phe Glu Met Phe Arg Glu Leu Asn Glu Ala Leu Glu Leu Lys Asp Ala
                        340 345 350
Gln Ala Gly Lys Glu Pro Gly Gly Ser Arg Ala His Ser Ser His Leu
                355 360 365
Lys Ser Lys Lys Gly Gln Ser Thr Ser Arg His Lys Lys Leu Met Phe
        370 375 380
Lys Thr Glu Gly Pro Asp Ser Asp
385 390
SEQ ID NO: 4-Heavy chain of 2D3A8 antibody
Glu Val Gln Leu Gln Gln Ser Gly Pro Glu Leu Val Lys Pro Gly Ala
1 5 10 15
Ser Val Lys Met Ser Cys Lys Ala Ser Gly Tyr Thr Phe Thr Ser Tyr
                        20 25 30
Val Met His Trp Val Lys Gln Lys Pro Gly Gln Gly Leu Glu Trp Ile
                35 40 45
Gly Tyr Ile Asn Pro Tyr Asn Asp Gly Thr Lys Tyr Asn Glu Lys Phe
        50 55 60
Lys Gly Lys Ala Thr Leu Thr Ser Asp Lys Ser Ser Ser Thr Ala Tyr
65 70 75 80
Met Glu Leu Ser Ser Leu Thr Ser Glu Asp Ser Ala Val Tyr Tyr Cys
                                85 90 95
Ala Arg Gly Gly Tyr Tyr Ala Met Asp Tyr Trp Gly Gln Gly Thr Ser
                        100 105 110
Val Thr Val Ser Ser Glu Ser Gln Ser Phe Pro Asn Val Phe Pro Leu
                115 120 125
Val Ser Cys Glu Ser Pro Leu Ser Asp Lys Asn Leu Val Ala Met Gly
        130 135 140
Cys Leu Ala Arg Asp Phe Leu Pro Ser Thr Ile Ser Phe Thr Trp Asn
145 150 155 160
Tyr Gln Asn Asn Thr Glu Val Ile Gln Gly Ile Arg Thr Phe Pro Thr
                                165 170 175
Leu Arg Thr Gly Gly Lys Tyr Leu Ala Thr Ser Gln Val Leu Leu Ser
                        180 185 190
Pro Lys Ser Ile Leu Glu Gly Ser Asp Glu Tyr Leu Val Cys Lys Ile
                195 200 205
His Tyr Gly Gly Lys Asn Arg Asp Leu His Val Pro Ile Pro Ala Val
        210 215 220
Ala Glu Met Asn Pro Asn Val Asn Val Phe Val Pro Pro Arg Asp Gly
225 230 235 240
Phe Ser Gly Pro Ala Pro Arg Lys Ser Lys Leu Ile Cys Glu Ala Thr
                                245 250 255
Asn Phe Thr Pro Lys Pro Ile Thr Val Ser Trp Leu Lys Asp Gly Lys
                        260 265 270
Leu Val Glu Ser Gly Phe Thr Thr Asp Pro Val Thr Ile Glu Asn Lys
                275 280 285
Gly Ser Thr Pro Gln Thr Tyr Lys Val Ile Ser Thr Leu Thr Ile Ser
        290 295 300
Glu Ile Asp Trp Leu Asn Leu Asn Val Tyr Thr Cys Arg Val Asp His
305 310 315 320
Arg Gly Leu Thr Phe Leu Lys Asn Val Ser Ser Thr Cys Ala Ala Ser
                                325 330 335
Pro Ser Thr Asp Ile Leu Thr Phe Thr Ile Pro Pro Ser Phe Ala Asp
                        340 345 350
Ile Phe Leu Ser Lys Ser Ala Asn Leu Thr Cys Leu Val Ser Asn Leu
                355 360 365
Ala Thr Tyr Glu Thr Leu Asn Ile Ser Trp Ala Ser Gln Ser Gly Glu
        370 375 380
Pro Leu Glu Thr Lys Ile Lys Ile Met Glu Ser His Pro Asn Gly Thr
385 390 395 400
Phe Ser Ala Lys Gly Val Ala Ser Val Cys Val Glu Asp Trp Asn Asn
                                405 410 415
Arg Lys Glu Phe Val Cys Thr Val Thr His Arg Asp Leu Pro Ser Pro
                        420 425 430
Gln Lys Lys Phe Ile Ser Lys Pro Asn Glu Val His Lys His Pro Pro
                435 440 445
Ala Val Tyr Leu Leu Pro Pro Ala Arg Glu Gln Leu Asn Leu Arg Glu
        450 455 460
Ser Ala Thr Val Thr Cys Leu Val Lys Gly Phe Ser Pro Ala Asp Ile
465 470 475 480
Ser Val Gln Trp Leu Gln Arg Gly Gln Leu Leu Pro Gln Glu Lys Tyr
                                485 490 495
Val Thr Ser Ala Pro Met Pro Glu Pro Gly Ala Pro Gly Phe Tyr Phe
                        500 505 510
Thr His Ser Ile Leu Thr Val Thr Glu Glu Glu Trp Asn Ser Gly Glu
                515 520 525
Thr Tyr Thr Cys Val Val Gly His Glu Ala Leu Pro His Leu Val Thr
        530 535 540
Glu Arg Thr Val Asp Lys Ser Thr Gly Lys Pro Thr Leu Tyr Asn Val
545 550 555 560
Ser Leu Ile Met Ser Asp Thr Gly Gly Thr Cys Tyr
                                565 570
SEQ ID NO: 5-Light chain of 2D3A8 antibody
Asp Ile Gln Met Thr Gln Thr Thr Ser Ser Leu Ser Ala Ser Leu Gly
1 5 10 15
Asp Arg Val Thr Ile Ser Cys Arg Ala Ser Gln Asp Ile Ser Asn Tyr
                        20 25 30
Leu Asn Trp Tyr Gln Gln Lys Pro Asp Gly Thr Val Lys Leu Leu Ile
                35 40 45
Tyr Tyr Thr Ser Arg Leu His Ser Gly Val Pro Ser Arg Phe Ser Gly
        50 55 60
Ser Gly Ser Gly Thr Asp Tyr Ser Leu Thr Ile Ser Asn Leu Glu Gln
65 70 75 80
Glu Asp Ile Ala Thr Tyr Phe Cys Gln Gln Gly Asn Thr Leu Pro Tyr
                                85 90 95
Thr Phe Gly Gly Gly Thr Lys Leu Glu Ile Lys Arg Ala Asp Ala Ala
                        100 105 110
Pro Thr Val Ser Ile Phe Pro Pro Ser Ser Glu Gln Leu Thr Ser Gly
                115 120 125
Gly Ala Ser Val Val Cys Phe Leu Asn Asn Phe Tyr Pro Lys Asp Ile
        130 135 140
Asn Val Lys Trp Lys Ile Asp Gly Ser Glu Arg Gln Asn Gly Val Leu
145 150 155 160
Asn Ser Trp Thr Asp Gln Asp Ser Lys Asp Ser Thr Tyr Ser Met Ser
                                165 170 175
Ser Thr Leu Thr Leu Thr Lys Asp Glu Tyr Glu Arg His Asn Ser Tyr
                        180 185 190
Thr Cys Glu Ala Thr His Lys Thr Ser Thr Ser Pro Ile Val Lys Ser
                195 200 205
Phe Asn Arg Asn Glu Cys
        210
SEQ ID NO: 6-Heavy chain variable region of 2D3A8 antibody
Glu Val Gln Leu Gln Gln Ser Gly Pro Glu Leu Val Lys Pro Gly Ala
1 5 10 15
Ser Val Lys Met Ser Cys Lys Ala Ser Gly Tyr Thr Phe Thr Ser Tyr
                        20 25 30
Val Met His Trp Val Lys Gln Lys Pro Gly Gln Gly Leu Glu Trp Ile
                35 40 45
Gly Tyr Ile Asn Pro Tyr Asn Asp Gly Thr Lys Tyr Asn Glu Lys Phe
        50 55 60
Lys Gly Lys Ala Thr Leu Thr Ser Asp Lys Ser Ser Ser Thr Ala Tyr
65 70 75 80
Met Glu Leu Ser Ser Leu Thr Ser Glu Asp Ser Ala Val Tyr Tyr Cys
                                85 90 95
Ala Arg Gly Gly Tyr Tyr Ala Met Asp Tyr Trp Gly Gln Gly Thr Ser
                        100 105 110
Val Thr Val Ser Ser
                115
SEQ ID NO: 7-Light chain variable region of 2D3A8 antibody
Asp Ile Gln Met Thr Gln Thr Thr Ser Ser Leu Ser Ala Ser Leu Gly
1 5 10 15
Asp Arg Val Thr Ile Ser Cys Arg Ala Ser Gln Asp Ile Ser Asn Tyr
                        20 25 30
Leu Asn Trp Tyr Gln Gln Lys Pro Asp Gly Thr Val Lys Leu Leu Ile
                35 40 45
Tyr Tyr Thr Ser Arg Leu His Ser Gly Val Pro Ser Arg Phe Ser Gly
        50 55 60
Ser Gly Ser Gly Thr Asp Tyr Ser Leu Thr Ile Ser Asn Leu Glu Gln
65 70 75 80
Glu Asp Ile Ala Thr Tyr Phe Cys Gln Gln Gly Asn Thr Leu Pro Tyr
                                85 90 95
Thr Phe Gly Gly Gly Thr Lys Leu Glu Ile Lys
                        100 105
SEQ ID NO: 8-Heavy chain CDR1 of 2D3A8 antibody
Ser Tyr Val Met His
1 5
SEQ ID NO: 9-Heavy chain CDR2 of 2D3A8 antibody
Tyr Ile Asn Pro Tyr Asn Asp Gly Thr Lys Tyr Asn Glu Lys Phe Lys
1 5 10 15
Gly
SEQ ID NO: 10-Heavy chain CDR3 of 2D3A8 antibody
Gly Gly Tyr Tyr Ala Met Asp Tyr
1 5
SEQ ID NO: 11-Light chain CDR1 of 2D3A8 antibody
Arg Ala Ser Gln Asp Ile Ser Asn Tyr Leu Asn
1 5 10
SEQ ID NO: 12-Light chain CDR2 of 2D3A8 antibody
Tyr Thr Ser Arg Leu His Ser
1 5
SEQ ID NO: 13-Light chain CDR3 of 2D3A8 antibody
Gln Gln Gly Asn Thr Leu Pro Tyr Thr
1 5
SEQ ID NO: 14-P1 peptide
Thr Glu Glu Glu Asn Leu Arg
SEQ ID NO: 15-P1 fragment
Glu Glu Asn Leu Arg
SEQ ID NO: 16-P1 fragment
Thr Glu
SEQ ID NO: 17-P1 fragment
Glu Asn Leu Arg
SEQ ID NO: 18-P1 fragment
Thr Glu Glu
SEQ ID NO: 19-P1 fragment
Leu Arg
SEQ ID NO: 20-P1 fragment
Thr Glu Glu Glu Asn
SEQ ID NO: 21-P1 fragment
Arg
SEQ ID NO: 22-P1 fragment
Glu Glu Glu Asn Leu Arg
SEQ ID NO: 23-P1 fragment
Thr Glu Glu Glu
SEQ ID NO: 24-P1 fragment
Asn Leu Arg
```

### Mass Spectrometry

Mass spectrometry (MS) is an analytical technique that is used to measure the mass-to-charge ratio of ions. The results are presented as a mass spectrum, a plot of intensity as a function of the mass-to-charge ratio. Mass spectrometry is used in many different fields and is applied to pure samples as well as complex mixtures.

A mass spectrum is a type of plot of the ion signal as a function of the mass-to-charge ratio. These spectra are used to determine the elemental or isotopic signature of a sample, the masses of particles and of molecules, and to elucidate the chemical identity or structure of molecules and other chemical compounds.

In a typical MS procedure, a sample, which may be solid, liquid, or gaseous, is ionized, for example by bombarding it with a beam of electrons. This may cause some of the sample's molecules to break up into positively charged fragments or simply become positively charged without fragmenting. These ions (fragments) are then separated according to their mass-to-charge ratio, for example by accelerating them and subjecting them to an electric or magnetic field: ions of the same mass-to-charge ratio will undergo the same amount of deflection or directional focusing (Sparkman, O. David (2000). Mass spectrometry desk reference. Pittsburgh: Global View Pub. ISBN 978-O-9660813-2-9). The ions are detected by a mechanism capable of detecting charged particles, such as an electron multiplier. Results are displayed as spectra of the signal intensity of detected ions as a function of the mass-to-charge ratio. The atoms or molecules in the sample can be identified by correlating known masses (e.g., an entire molecule) to the identified masses or through a characteristic fragmentation pattern.

### Components of Mass Spectrometer

A mass spectrometer consists of three components: an ion source, a mass analyzer, and a detector. The ionizer converts a portion of the sample into ions. There is a wide variety of ionization techniques, depending on the phase (solid, liquid, gas) of the sample and the efficiency of various ionization mechanisms for the unknown species. An extraction system removes ions from the sample, which are then targeted through the mass analyzer and into the detector. The differences in masses of the fragments allows the mass analyzer to sort the ions by their mass-to-charge ratio. The detector measures the value of an indicator quantity and thus provides data for calculating the abundances of each ion present. Some detectors also give spatial information, e.g., a multichannel plate.

### Ionization

The ion source is the part of the mass spectrometer that ionizes the material under analysis (the analyte). The ions are then transported by magnetic or electric fields to the mass analyzer.

Two techniques often used with liquid and solid biological samples include electrospray ionization (Fenn J B, Mann M, Meng C K, Wong S F, Whitehouse C M (October 1989). “Electrospray ionization for mass spectrometry of large biomolecules”. Science. 246 (4926)) and matrix-assisted laser desorption/ionization (MALDI) (Tanaka K, Waki H, Ido Y, Akita S, Yoshida Y, Yoshida T (1988). “Protein and Polymer Analyses up to m/z 100 000 by Laser Ionization Time-of flight Mass Spectrometry”. Rapid Commun Mass Spectrom. 2 (20): 151-3.)

In mass spectrometry, ionization refers to the production of gas phase ions suitable for resolution in the mass analyzer or mass filter. Ionization occurs in the ion source. There are several ion sources available; each has advantages and disadvantages for particular applications. For example, electron ionization (EI) gives a high degree of fragmentation, yielding highly detailed mass spectra which when skillfully analyzed can provide important information for structural elucidation/characterization and facilitate identification of unknown compounds by comparison to mass spectral libraries obtained under identical operating conditions. However, EI is not suitable for coupling to HPLC, LC-MS, since at atmospheric pressure, the filaments used to generate electrons burn out rapidly. Thus, EI is coupled predominantly with GC, GC-MS, where the entire system is under high vacuum.

### Hard Ionization and Soft Ionization

Hard ionization techniques are processes which impart high quantities of residual energy in the subject molecule invoking large degrees of fragmentation (i.e., the systematic rupturing of bonds acts to remove the excess energy, restoring stability to the resulting ion). Resultant ions tend to have m/z lower than the molecular ion (other than in the case of proton transfer and not including isotope peaks). The most common example of hard ionization is electron ionization (EI).

Soft ionization refers to the processes which impart little residual energy onto the subject molecule and as such result in little fragmentation. Examples include fast atom bombardment (FAB), chemical ionization (CI), atmospheric-pressure chemical ionization (APCI), atmospheric-pressure photoionization (APPI), electrospray ionization (ESI), desorption electrospray ionization (DESI), and matrix-assisted laser desorption/ionization (MALDI).

### Inductively Coupled Plasma

Inductively coupled plasma (ICP) sources are used primarily for cation analysis of a wide array of sample types. In this source, a plasma that is electrically neutral overall, but that has had a substantial fraction of its atoms ionized by high temperature, is used to atomize introduced sample molecules and to further strip the outer electrons from those atoms. The plasma is usually generated from argon gas, since the first ionization energy of argon atoms is higher than the first of any other elements except, He, F and Ne, but lower than the second ionization energy of all except the most electropositive metals. The heating is achieved by a radio-frequency current passed through a coil surrounding the plasma.

### Photoionization Mass Spectrometry

Photoionization can be used in experiments which seek to use mass spectrometry as a means of resolving chemical kinetics mechanisms and isomeric product branching. [14] In such instances a high energy photon, either X-ray or UV, is used to dissociate stable gaseous molecules in a carrier gas of He or Ar. In instances where a synchrotron light source is utilized, a tunable photon energy can be utilized to acquire a photoionization efficiency curve which can be used in conjunction with the charge ratio m/z to fingerprint molecular and ionic species. More recently atmospheric pressure photoionization (APPI) has been developed to ionize molecules mostly as effluents of LC-MS systems.

### Ambient Ionization

Some applications for ambient ionization include environmental applications as well as clinical applications. In these techniques, ions form in an ion source outside the mass spectrometer. Sampling becomes easy as the samples don't need previous separation nor preparation. Some examples of ambient ionization techniques are Direct Analysis in Real Time (DART), DESI, SESI, LAESI, desorption atmospheric-pressure chemical ionization (DAPCI), and desorption atmospheric pressure photoionization DAPPI among others.

### Other Ionization Techniques

Others include glow discharge, field desorption (FD), fast atom bombardment (FAB), thermospray, desorption/ionization on silicon (DIOS), atmospheric pressure chemical ionization (APCI), secondary ion mass spectrometry (SIMS), spark ionization and thermal ionization (TIMS). (Bruins, A. P. (1991). “Mass spectrometry with ion sources operating at atmospheric pressure”. Mass Spectrometry Reviews. 10 (1): 53-77).

### Mass Selection

Mass analyzers separate the ions according to their mass-to-charge ratio.

The differential equation is the classic equation of motion for charged particles. Together with the particle's initial conditions, it completely determines the particle's motion in space and time in terms of m/Q. Thus mass spectrometers could be thought of as “mass-to-charge spectrometers”. When presenting data, it is common to use the (officially) dimensionless m/z, where z is the number of elementary charges (e) on the ion (z=Q/e). This quantity, although it is informally called the mass-to-charge ratio, more accurately speaking represents the ratio of the mass number and the charge number, z.

There are many types of mass analyzers, using either static or dynamic fields, and magnetic or electric fields, but all operate according to the above differential equation. Each analyzer type has its strengths and weaknesses. Many mass spectrometers use two or more mass analyzers for tandem mass spectrometry (MS/MS). In addition to the more common mass analyzers listed below, there are others designed for special situations.

There are several important analyzer characteristics. The mass resolving power is the measure of the ability to distinguish two peaks of slightly different m/z. The mass accuracy is the ratio of the m/z measurement error to the true m/z. Mass accuracy is usually measured in ppm or milli mass units. The mass range is the range of m/z amenable to analysis by a given analyzer. The linear dynamic range is the range over which ion signal is linear with analyte concentration. Speed refers to the time frame of the experiment and ultimately is used to determine the number of spectra per unit time that can be generated.

### Time-of-Flight Mass Spectrometry

The time-of-flight (TOF) analyzer uses an electric field to accelerate the ions through the same potential, and then measures the time they take to reach the detector. If the particles all have the same charge, their kinetic energies will be identical, and their velocities will depend only on their masses. Ions with a lower mass will reach the detector first. (Wollnik, H. (1993). “Time-of-flight mass analyzers”. Mass Spectrometry Reviews. 12 (2): 89-114.).

However, in reality, even particles with the same m/z can arrive at different times at the detector, because they have different initial velocities. The initial velocity is often not dependent on the mass of the ion and will turn into a difference in the final velocity. Because of this, ions with the same m/z ratio will reach the detector at a variety of times, which broadens the peaks shown on the count vs m/z plot but will generally not change the central location of the peaks, since the starting velocity of ions is generally centered at zero. To fix this problem, time-lag focusing/delayed extraction has been coupled with TOF-MS. (Guilhaus M (1998). “Principles and Instrumentation in Time-of-flight Mass Spectrometry” Journal of Mass Spectrometry. 30 (11): 1519-1532.)

### Quadrupole Mass Filter

Quadrupole mass analyzers use oscillating electrical fields to selectively stabilize or destabilize the paths of ions passing through a radio frequency (RF) quadrupole field created between four parallel rods. Only the ions in a certain range of mass/charge ratio are passed through the system at any time, but changes to the potentials on the rods allow a wide range of m/z values to be swept rapidly, either continuously or in a succession of discrete hops. A quadrupole mass analyzer acts as a mass-selective filter and is closely related to the quadrupole ion trap, particularly the linear quadrupole ion trap except that it is designed to pass the untrapped ions rather than collect the trapped ones, and is for that reason referred to as a transmission quadrupole. A magnetically enhanced quadrupole mass analyzer includes the addition of a magnetic field, either applied axially or transversely. This novel type of instrument leads to an additional performance enhancement in terms of resolution and/or sensitivity depending upon the magnitude and orientation of the applied magnetic field. (Syed S U, Maher S, Taylor S (December 2013). “Quadrupole mass filter operation under the influence of magnetic field”. Journal of Mass Spectrometry. 48 (12): 1325-39; Maher S, Syed S U, Hughes D M, Gibson J R, Taylor S (August 2013). “Mapping the stability diagram of a quadrupole mass spectrometer with a static transverse magnetic field applied”. Journal of the American Society for Mass Spectrometry. 24 (8): 1307-14.) A common variation of the transmission quadrupole is the triple quadrupole mass spectrometer. The “triple quad” has three consecutive quadrupole stages, the first acting as a mass filter to transmit a particular incoming ion to the second quadrupole, a collision chamber, wherein that ion can be broken into fragments. The third quadrupole also acts as a mass filter, to transmit a particular fragment ion to the detector. If a quadrupole is made to rapidly and repetitively cycle through a range of mass filter settings, full spectra can be reported. Likewise, a triple quad can be made to perform various scan types characteristic of tandem mass spectrometry.

### Three-Dimensional Quadrupole Ion Trap

The quadrupole ion trap works on the same physical principles as the quadrupole mass analyzer, but the ions are trapped and sequentially ejected. Ions are trapped in a mainly quadrupole RF field, in a space defined by a ring electrode (usually connected to the main RF potential) between two endcap electrodes (typically connected to DC or auxiliary AC potentials). The sample is ionized either internally (e.g., with an electron or laser beam), or externally, in which case the ions are often introduced through an aperture in an endcap electrode.

There are many mass/charge separation and isolation methods but the most commonly used is the mass instability mode in which the RF potential is ramped so that the orbit of ions with a mass a>b are stable while ions with mass b become unstable and are ejected on the z-axis onto a detector. There are also non-destructive analysis methods.

Ions may also be ejected by the resonance excitation method, whereby a supplemental oscillatory excitation voltage is applied to the endcap electrodes, and the trapping voltage amplitude and/or excitation voltage frequency is varied to bring ions into a resonance condition in order of their mass/charge ratio. (Paul W, Steinwedel H (1953). “Ein neues Massenspektrometer ohne Magnetfeld”. Zeitschrift fir Naturforschung A. 8 (7): 448-450.; Paul W, Steinwedel H (1953). “Ein neues Massenspektrometer ohne Magnetfeld”. Zeitschriftfiir Naturforschung A. 8 (7): 448-450.)

### Cylindrical Ion Trap

The cylindrical ion trap mass spectrometer (CIT) is a derivative of the quadrupole ion trap where the electrodes are formed from flat rings rather than hyperbolic shaped electrodes. The architecture lends itself well to miniaturization because as the size of a trap is reduced, the shape of the electric field near the center of the trap, the region where the ions are trapped, forms a shape similar to that of a hyperbolic trap.

### Linear Quadrupole Ion Trap

A linear quadrupole ion trap is similar to a quadrupole ion trap, but it traps ions in a two dimensional quadrupole field, instead of a three-dimensional quadrupole field as in a 3D quadrupole ion trap. Thermo Fisher's LTQ (“linear trap quadrupole”) is an example of the linear ion trap. (Schwartz J C, Senko M W, Syka J E (June 2002). “A two-dimensional quadrupole ion trap mass spectrometer”. Journal of the American Society for Mass Spectrometry. 13 (6): 659-69.)

A toroidal ion trap can be visualized as a linear quadrupole curved around and connected at the ends or as a cross-section of a 3D ion trap rotated on edge to form the toroid, donut-shaped trap. The trap can store large volumes of ions by distributing them throughout the ring-like trap structure. This toroidal shaped trap is a configuration that allows the increased miniaturization of an ion trap mass analyzer. Additionally, all ions are stored in the same trapping field and ejected together simplifying detection that can be complicated with array configurations due to variations in detector alignment and machining of the arrays. (Schwartz J C, Senko M W, Syka J E (June 2002). “A two-dimensional quadrupole ion trap mass spectrometer”. Journal of the American Society for Mass Spectrometry. 13 (6): 659-69.)

As with the toroidal trap, linear traps and 3D quadrupole ion traps are the most commonly miniaturized mass analyzers due to their high sensitivity, tolerance for mTorr pressure, and capabilities for single analyzer tandem mass spectrometry (e.g. product ion scans). (Snyder D T, Pulliam C J, Ouyang Z, Cooks R G (January 2016). “Miniature and Fieldable Mass Spectrometers: Recent Advances”. Analytical Chemistry. 88 (1): 2-29.)

### Orbitrap

Orbitrap instruments are similar to Fourier-transform ion cyclotron resonance mass spectrometers (see text below). Ions are electrostatically trapped in an orbit around a central, spindle shaped electrode. The electrode confines the ions so that they both orbit around the central electrode and oscillate back and forth along the central electrode's long axis. This oscillation generates an image current in the detector plates which is recorded by the instrument. The frequencies of these image currents depend on the mass-to-charge ratios of the ions. Mass spectra are obtained by Fourier transformation of the recorded image currents. Orbitraps have a high mass accuracy, high sensitivity and a good dynamic range. (Hu Q, Noll R J, Li H, Makarov A, Hardman M, Graham Cooks R (April 2005). “The Orbitrap: a new mass spectrometer”. Journal of Mass Spectrometry.)

### Fourier-Transform Mass Spectrometry (FTMS)

Fourier-transform mass spectrometry (FTMS), or more precisely Fourier-transform ion cyclotron resonance MS, measures mass by detecting the image current produced by ions cyclotroning in the presence of a magnetic field. Instead of measuring the deflection of ions with a detector such as an electron multiplier, the ions are injected into a Penning trap (a static electric/magnetic ion trap) where they effectively form part of a circuit. Detectors at fixed positions in space measure the electrical signal of ions which pass near them over time, producing a periodic signal. Since the frequency of an ion's cycling is determined by its mass-to-charge ratio, this can be deconvoluted by performing a Fourier transform on the signal. FTMS has the advantage of high sensitivity (since each ion is “counted” more than once) and much higher resolution and thus precision (Comisarow M B, Marshall A G (1974). “Fourier-transform ion cyclotron resonance spectroscopy”. Chemical Physics Letters. 25 (2): 282-283; Comisarow M B, Marshall A G (1974). “Fourier-transform ion cyclotron resonance spectroscopy”. Chemical Physics Letters. 25 (2): 282-283.)

Ion cyclotron resonance (ICR) is an older mass analysis technique similar to FTMS except that ions are detected with a traditional detector. Ions trapped in a Penning trap are excited by an RF electric field until they impact the wall of the trap, where the detector is located. Ions of different mass are resolved according to impact time.

### Detectors

The final element of the mass spectrometer is the detector. The detector records either the charge induced or the current produced when an ion passes by or hits a surface. In a scanning instrument, the signal produced in the detector during the course of the scan versus where the instrument is in the scan (at what m/Q) will produce a mass spectrum, a record of ions as a function of m/Q.

Typically, some type of electron multiplier is used, though other detectors including Faraday cups and ion-to-photon detectors are also used. Because the number of ions leaving the mass analyzer at a particular instant is typically quite small, considerable amplification is often necessary to get a signal. Microchannel plate detectors are commonly used in modern commercial instruments. (Dubois F, Knochenmuss R, Zenobi R, Brunelle A, Deprun C, Le Beyec Y (1999). “A comparison between ion-to-photon and microchannel plate detectors”. Rapid Communications in Mass Spectrometry. 13 (9): 786-791). In FTMS and Orbitraps, the detector consists of a pair of metal surfaces within the mass analyzer/ion trap region which the ions only pass near as they oscillate. No direct current is produced, only a weak AC image current is produced in a circuit between the electrodes. Other inductive detectors have also been used. (Park M A, Callahan J H, Vertes A (1994). “An inductive detector for time-of-flight mass spectrometry”. Rapid Communications in Mass Spectrometry. 8 (4): 317-322).

### Interpretation of Mass Spectra

Mass spectrometry is an important method for the characterization and sequencing of proteins. The two primary methods for ionization of whole proteins are electrospray ionization (ESI) and matrix-assisted laser desorption/ionization (MALDI). In keeping with the performance and mass range of available mass spectrometers, two approaches are used for characterizing proteins.

In the first, intact proteins are ionized by either of the two techniques described above, and then introduced to a mass analyzer. This approach is referred to as “top-down” strategy of protein analysis. The top-down approach however is largely limited to low-throughput single-protein studies. In the second, proteins are enzymatically digested into smaller peptides using proteases such as trypsin or pepsin, either in solution or in gel after electrophoretic separation. Other proteolytic agents are also used.

The collection of peptide products are often separated by chromatography prior to introduction to the mass analyzer. When the characteristic pattern of peptides is used for the identification of the protein the method is called peptide mass fingerprinting (PMF), if the identification is performed using the sequence data determined in tandem MS analysis it is called de novo peptide sequencing. These procedures of protein analysis are also referred to as the “bottom-up” approach and have also been used to analyze the distribution and position of post-translational modifications such as phosphorylation on proteins. (Ferries S, Perkins S, Brownridge P J, Campbell A, Eyers P A, Jones A R, Eyers C E (September 2017). “Evaluation of Parameters for Confident Phosphorylation Site Localization Using an Orbitrap Fusion Tribrid Mass Spectrometer”. Journal of Proteome Research. 16 (9): 3448-3459) A third approach is also beginning to be used, this intermediate “middle-down” approach involves analyzing proteolytic peptides that are larger than the typical tryptic peptide.

Mass spectrometry produces various types of data. The most common data representation is the mass spectrum. Certain types of mass spectrometry data are best represented as a mass chromatogram. Types of chromatograms include selected ion monitoring (SIM), total ion current (TIC), and selected reaction monitoring (SRM), among many others. Other types of mass spectrometry data are well represented as a three-dimensional contour map. In this form, the mass-to-charge, m/z is on the x-axis, intensity the y-axis, and an additional experimental parameter, such as time, is recorded on the z-axis.

Since the precise structure or peptide sequence of a molecule is deciphered through the set of fragment masses, the interpretation of mass spectra requires combined use of various techniques. Usually, the first strategy for identifying an unknown compound is to compare its experimental mass spectrum against a library of mass spectra. If no matches result from the search, then manual interpretation (Tureček F, McLafferty F W (1993). Interpretation of mass spectra. Sausalito: University Science Books. ISBN 978-O-935702-25-5) or software assisted interpretation of mass spectra must be performed. Computer simulation of ionization and fragmentation processes occurring in mass spectrometer is the primary tool for assigning structure or peptide sequence to a molecule. An a priori structural information is fragmented in silico and the resulting pattern is compared with observed spectrum. Such simulation is often supported by a fragmentation library (Mistrik, R. (2004). A New Concept for the Interpretation of Mass Spectra Based on a Combination of a Fragmentation Mechanism Database and a Computer Expert System.in Ashcroft, A. E., Brenton, G., Monaghan, J. J. (Eds.), Advances in Mass Spectrometry, Elsevier, Amsterdam, vol. 16, pp. 821.) that contains published patterns of known decomposition reactions. Software taking advantage of this idea has been developed for both small molecules and proteins.

Analysis of mass spectra can also be spectra with accurate mass. A mass-to-charge ratio value (m/z) with only integer precision can represent an immense number of theoretically possible ion structures; however, more precise mass figures significantly reduce the number of candidate molecular formulas. A computer algorithm called formula generator calculates all molecular formulas that theoretically fit a given mass with specified tolerance.

A recent technique for structure elucidation in mass spectrometry, called precursor ion fingerprinting, identifies individual pieces of structural information by conducting a search of the tandem spectra of the molecule under investigation against a library of the product-ion spectra of structurally characterized precursor ions. (Sheldon M T, Mistrik R, Croley T R (March 2009). “Determination of ion structures in structurally related compounds using precursor ion fingerprinting”. Journal of the American Society for Mass Spectrometry)

### MATERIALS AND METHODS

### Materials

Antibody-based plasma/serum depletion Seppro IgY14 LC10 column was purchased from Sigma-Aldrich (Merck KGaA, Darmstadt, Germany). Trypsin (Sequencing grade modified porcine) was obtained from Promega. Acetonitrile was purchased from JT Baker, and formic acid was obtained from EMD Millipore (Billerica, MA, USA). C18 Cartridges for sample preparation, and chromatography columns for reverse phase HPLC and online HPLC of Triple Quadrupole mass spectrometer were purchased from Waters (Milford, Massachusetts). Plasma Dilution Buffer, -Plasma Washing Buffer and -Plasma Elution Buffer, were prepared following standard protocols for this method. All other reagents were purchased from Sigma-Aldrich (St. Louis, Missouri) unless otherwise indicated.

### Labelled U-p53 Isoform Protein (Conformational Variant) Produced by Nitrosilation Reaction

SIN-1 is a metabolite of molsidomine which spontaneously releases nitric oxide and superoxide anion which react to form peroxynitrite under physiological conditions. (Nishikawa et al. (1982) Inhibition of platelet aggregation and stimulation of guanylate cyclase by an antianginal agent molsidomine and its metabolites; J. Pharmacol. Exp. Ther. 220). SIN-1 was used for nitrosilation reactions. SIN-1 was resuspended in 50 ul bi-distilled water.

The conformational variant of p53 (U-p53) was generated by a nitrosilation reaction of the 10 cysteine residues using SIN-1 in a ratio of 9:1 (e.g., 45 μL of SIN-1 are mixed with 5 μL of the p53 protein) and then incubated for 30 min at 37° C. in the dark. After the reaction, the U-p53 conformational variant was aliquoted and stored at −20° C.

Buffer A: Tris 25 mM, Sodium Chloride (NaCl) 0.15 mM, Tween-20 50 mM (E.g.: 303 mg of Tris, 87.66 mg of NaCL was dissolved in 100 ml of bi-distilled water for a 100× stock solution. 6.14 g of Tween-20 was solubilized first in 89 mL of bi-distilled water and 1 ml of the 100× NaCl was added and then the volume was made up to 100 mL by adding bi-distilled water).

Buffer B: Glycine 0.1 M pH 2.0: 750 mg of glycine are solubilized first in 90 mL of bi-distilled water then a final volume of 100 mL was then obtained by adding bi-distilled water. HCl 5 Normal was used to obtain a final pH value of 2.

Ammonium Bicarbonate (AmBic) Digestion Buffer 50 mMoL pH 7.8: 0.4 g of AmBic was solubilised in 100 mL of bi-distilled.

Dithiothreitol (DTT) 180 mM in AmBic 50 mM: 0.3 g of DTT was solubilized in 10 mL of 50 mM AmBic. The solution was vortexed until the DTT was completely dissolved.

Iodoacetamide (IAA) 380 mM in AmBic 50 mM. Preparation: 0.7 g IAA was solubilized in 10 mL of 50 mM AmBic. The solution was vortexed until the IAA was completely dissolved.

Trypsin 25 ng/μL trypsin solution. Preparation: 20 μg of trypsin was solubilized in 800 μL of AmBic 50 mM solution. The solution was vortexed until the trypsin is completely dissolved and was aliquoted and stored at −20° C.

### Bead-Antibody Binding

Protein magnetic bead L 50 μL (0.5 mg) was collected in a Vial. 150 μL Buffer A was then added and vortex was applied. Magnetic surface was used to discard the supernatant. Buffer A 1 mL was added. Vortex was applied for 1 minute. Magnetic surface was used to discard the supernatant. Antibody solution (200 |μL, 0.05 μg/μL corresponding to 10 μg) was added to Protein L magnetic bead. The solution was mixed for 2 hours. Magnetic surface was used to discard the supernatant. Buffer A 500 μL was added. Magnetic surface was again used to discard the supernatant. Washing was repeated again, and the supernatant was discarded leaving behind the magnetic beads connected to antibody which was then reconstituted with 1 mL of Buffer A and stored at room temperature.

### Plasma Sample Depletion to Form Processed Sample

Blood plasma samples extracted from the different categories of subjects were thawed at room temperature under laminar flow cabinet for 30 min. The sample was split in 25 μL aliquots sufficient to perform triplicates which were separately processed. The remaining material was stored at −20° C. for retesting purpose.

The most abundant proteins in the plasma was depleted using a Seppro IgY14 column. However, the plasma depletion process does not remove the unfolded p53 protein (p53 isoform). Plasma samples were diluted 5× in Plasma Dilution buffer, filtered (0.22 μm), then injected into IgY LC10 columns attached to an Agilent 1200 HPLC system. The unretained fraction was collected. High-abundance proteins were eluted using Plasma Elution buffer.

In some instances, 5 μL of CH₃CN was added to 25 μL of plasma. The acetonitrile spike was repeated every 1 minute since to reach a mixture volume of 50 μL. Vortex was applied for 5 minutes until a white deposit is observed. The sample centrifugation took place at 13000 g for 10 minutes. 40 μL of supernatant was added to the bead-antibody complex. Vortex was then weakly applied. The mixture was incubated at room temperature for 1 hour and then at 4° C. overnight. A magnetic surface was used to remove the supernatant. 500 μl of Buffer A were added and the mixture was vortexed. A magnetic plane was used to remove the supernatant. 45 μl of Buffer B was added to the pellet and was incubated for 10 minutes at room temperature to form the processed sample, which was then subjected to beaded antibody binding, followed by elution of p53 isoform and then enzymatic digest of the same.

### Immunoprecipitation (IP)

Conjugation of antibodies to beads was optimized and performed using buffers as noted above. Briefly the monoclonal antibody specific for unfolded p53 protein was added directly to Protein G Dynal Magnetic Beads (as obtained from Thermofischer), and the antibody was bound to the beads in a buffer on a rotator at room temperature for 2 hrs. The antibody-bound beads then were washed by incubation in 50 mL Link-A buffer and collected on a magnet. The antibody was cross-linked to the protein G on the beads by incubation with 50 mL B buffer on a rotator at room temperature for 1 hr. The beads were then washed twice with 50 mL C buffer, resuspended in 50 mL C buffer, and rotated at room temperature for 15 min.

200 μl of processed sample (plasma depleted sample) was used for IP reactions, each sample is reconstituted with IP buffer into a 3 ml system and 3 ml antibody conjugated beads (1:1 volume ratio) were added to the system. The IP systems were incubated on a rotator with a speed of 32 rounds per minute at 4° C. for 18 hours. Beads were collected on a magnet and washed by IP-Wash buffer at 4° C. for 3 times, followed by elution of the antigen through IP-Elution buffer. Flow-through fractions were collected to form the eluate containing the p53 isoform or unfolded p53 protein.

### Enzymatic Digestion of the Eluted Unfolded p53 Protein (p53 Isoform)

Two aliquots of 15 μl of Dithiothreitol (DTT) 180 mM are added to 40 μL of the eluate obtained from the prior immunoprecipitation step. The mixture was incubated for 15 min at 50° C. and at room temperature for 30 minutes. Two aliquots of 15 μl of Iodoacetamide (IAA) at 400 mM were added. The obtained mixture was incubated for 15 minutes at room temperature. 1 μL of trypsin (25 ng/μL) containing Lys-c (50 ng/μL) and AmBic 50 mM is added to 46.45 μL of the obtained mixture. Incubation took place at 37° C. for 3.5 hours followed by 57° C. for 30 minutes. 1 μL of Formic Acid (HCOOH) was added to 47.45 μL of the obtained mixture to stop the enzymatic digestion. pH value was checked, and it was adjusted to be in the range 1-4. If it was higher than 4, progressive volume (1 μL) of Formic Acid was added to obtain a pH value between 1 and 4. 10 μL of the obtained mixture was then analyzed. The obtained mixture contained one or more in vitro generated proteolytic peptides such as P1 peptide derived from the unfolded U-p53 protein upon enzymatic digestion.

### Detection and Quantitation of P1 Peptide and P1 Peptide Fragments Using Mass Spectrometry

HPLC Ultimate 3000 (Thermofisher, USA) with a Phenomenex Kinetex PFP 50×4.1 mm 2.6 pm were used to perform the chromatographic analysis. Binary gradient was used: Phase A (H2O+O0.2% Formic Acid (HCOOH)) and Phase C acetonitrile (CH3CN). The gradient was reported in the table below. 10 μL of the obtained mixture from the enzymatic digest step noted above, was injected.

A suitable mass spectrometer for detecting peptide and peptide fragments such as LTQ Orbitrap XL or triple quad mass spectrometer was used for the data acquisition. SACI ionization source was employed. The potential surface was 47 V, Gas nebulizer pressure was 75 Psi and dry gas flow was 1.0 L/min. 350° C. of nebulizer temperature was employed together with 320° C. of dry gas one. SACI peptide adduct profile mode was employed for data acquisition (Cristoni et al. Rapid Commun Mass Spectrom. 2003; 17(17): 1973-81.).

### EXAMPLES

The following examples further illustrate aspects of the present invention. However, they are in no way a limitation of the teachings or disclosure of the present invention as set forth herein.

### Example 1—Generation of P1 Fragments

A biological sample (blood plasma) was first obtained from the subject. The sample can also be one of blood, saliva, CSF, plasma, or serum. The sample from the subject was then depleted from abundant proteins according to the protocols reported above. The test samples were then immunoprecipitated with the antibody (2D3A8) specific for isoform of p53 protein (unfolded p53 protein), enzyme digested and subjected to mass spec. The P1 peptide gets fragmented when exposed to the ionization source of the mass spectrometer.

### Example 2—Generation of Mass Spectra of P1 Fragments

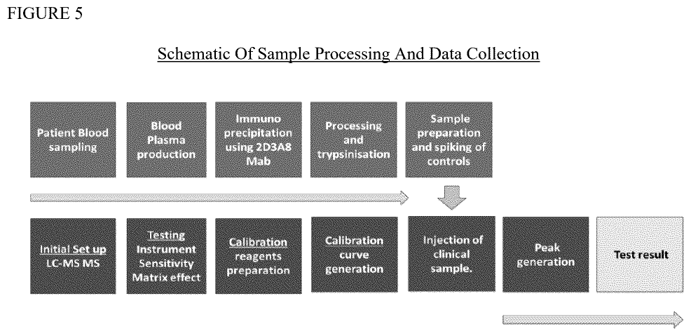

Mass spectrometry analysis of biological sample from a subject was performed according to the protocols listed below. FIG. 5 provides a schematic overview of the steps involved in processing the sample and performing mass spec. The biological sample from the subject was processed as noted in Example 1. The test sample was then spiked with synthetic labeled P1 peptide as internal control and used for mass spec analysis.

### Mass Spectrometer Tuning

A tuning sample, unlabeled P1 peptide (TEEENLR) was diluted to a suitable concentration in 0.2% v/v formic acid (in water). This tuning sample was used for initial tuning of mass spectrometer. Tuning concentration of 0.1 μg/ml was generally used for full scan analysis and for multiple reaction monitoring (MRM).

### Source Tuning

Using tuning standard prepared above, the source was set to standard conditions to obtain a stable aqueous spray. Scan was initiated between 400 and 1800 amu using 5-10p/min infusion. The source temperature and capillary voltage was adjusted to de-cluster dimers and increase the ion abundance of the MH²⁺ ion (m/z 445.9).

### Mass Analyzer Tuning

The mass spectrometer was set in single ion mode and the ion current for MH²⁺ ion was checked. The tuning was repeated to optimize the ion current. MRM was optimized for 445.5 (MH2+) to 660 (single charge) transition with the optimum mass window and dwell time. The collision gas was set to normal setting and the transport lens was also set to normal and autotune was allowed to optimize MS-MS fragmentation for the above transition.

### Mass Spectra

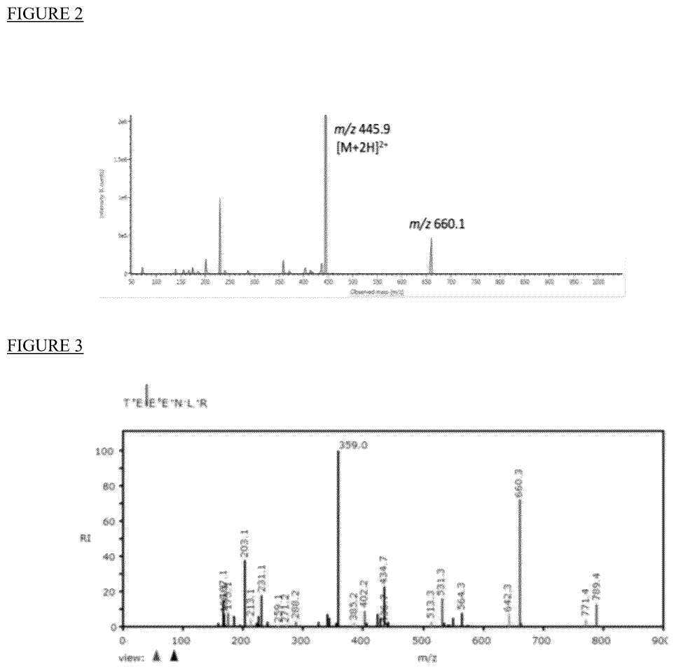

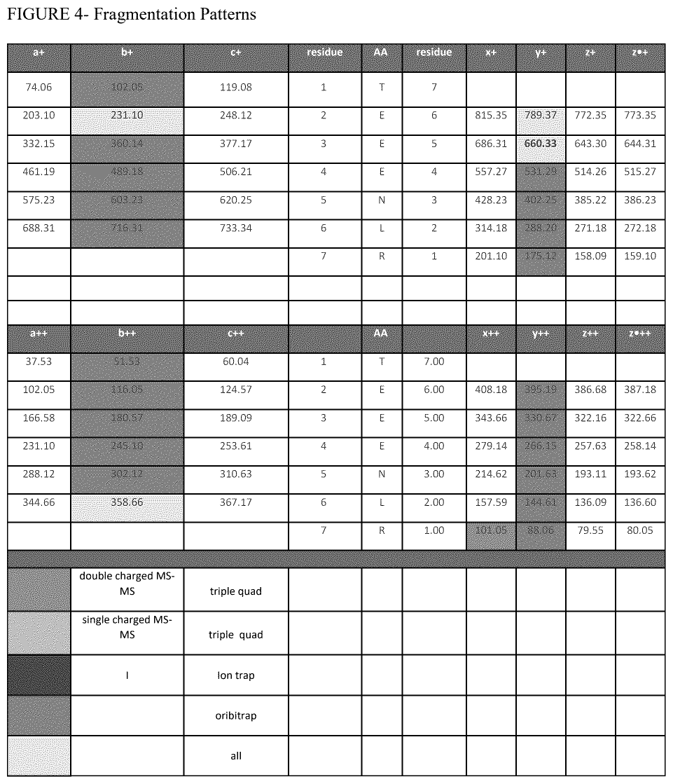

FIG. 2 shows the mass spectra of an exemplary sample showing two major fragments having m/z values of 660 and 231. The full-scale mass spectra of the test sample shows the presence of one or more peaks indicating the presence of one or more fragments. (See FIG. 3.) Two major peaks were observed in addition to the full-length peptide peak. The full-length peptide peak P1 had a m/z value of 445. The first major peak had a m/z value of 660 and the second major peak had a m/z value of 231. FIG. 4 shows a table listing fragments of P1 peptide from an exemplary sample along with mass and charge values. One can thus obtain mass spectrum of P1 fragments from the test sample.

### Example 3—Identification of P1 Fragments

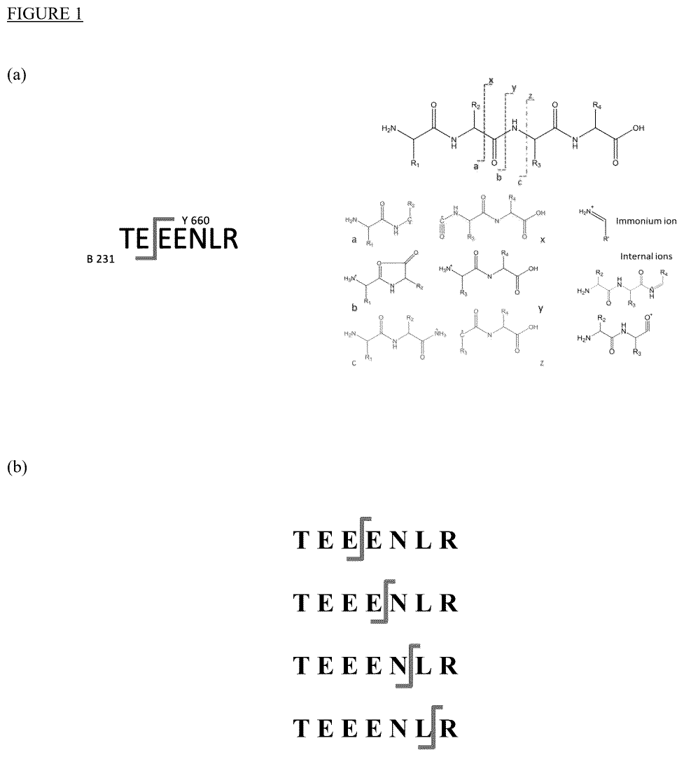

Briefly the sample was processed as noted in Example 1. The sample was then subjected to mass spectrometry as noted in Example 2. The identity of the P1 fragments generated from the sample was determined using Deep sequencing mass spectrometric analysis by Orbitrap MS. An exemplary listing of fragments generated during mass spectrometry run is shown in FIGS. 1a and 1b. The fragmentation pattern is also further exemplified in FIG. 4.

Deep sequence analysis revealed that the m/z peak at 660 corresponds to fragment-EENLR and the m/z peak at 231 corresponds to fragment-TE. The amount of each fragment and the P1 peptide was quantitated using internal control and standard curves. It was found that the amount of P1 peptide (precursor ion) and the fragments correlated with the amount of unfolded p53 protein in the subject.

Since the presence of these unique fragments of P1 peptide is directly corelated to the presence of unfolded p53 protein in the sample from the subject. One can use this method to determine whether unfolded p53 protein is present or absent in a given sample. The presence of these P1 fragments therefore signals the likelihood of presence of neurodegenerative disease such as Alzheimer's disease in the subject being tested.

### Example 4—Quantitation of P1 Fragments

Briefly the sample was processed as noted in Example 1. The sample was then subjected to mass spectrometry as noted in Example 2. The identity of the fragments generated from the P1 peptide was ascertained using the Deep sequencing analysis as noted in Example 3. The following example discloses the protocol for quantitation of the identified fragments of P1 peptide from the mass spectra.

### Calibration Curve

Unlabeled synthetic P1 peptide (889.9 Da) was used to create the dilution curve. The P1 peptide was suspended in 40 μL of water to obtain a solution of 0.5 fmol/μL. The P1 peptide control sample was then serial diluted in 0.2% v/v formic acid/water to give a suitable set of calibrators to cover the assay range (0.5 fmol/μL to 0.008 fmol/μL).

### Analysis

For each analytical run of test sample, at least three control samples were used. These control samples were obtained by spiking negative plasma with 0.05, 0.1, 0.2 fmol/μl of U-p53 labelled protein (as prepared in materials section) followed by immunoprecipitation and trypsinization. The test sample and the control sample were subjected to mass spectrometry as disclosed in Example 3 to generate data.

The results of the assay for the concentration of unfolded U-p53 protein were calculated comparing the peak area of labeled internal standard with peak area of each unlabeled P1 peptide analyte. The quantitation of P1 peptide in the clinical sample was obtained taking into consideration the peak area of the analyte vs peak area of the labelled standard peptide spikes in the sample.
The ratio between the peak area of the peptide in the clinical sample and the peak area of labelled standard peptide spiked in the sample (“A”) was calculated. The labeled P1 control peptide produces fragments (m/z 450 instead of 440 for labeled full length peptide; m/z of 670 for labeled EENLR fragment from labeled P1 peptide) with distinct m/z values that are different from those of the P1 peptide from the sample. Therefore, it was easily distinguishable from the sample peaks.
Calibration curve was created as described above.
The concentration of P1 peptide in each sample was calculated using the standard curve formula.

Briefly, the P1 peptide quantitation in the clinical sample was obtained by the interpolation of the area ratio (“A”) of the sample in the standard curve (formula: Y=mX+c): the area ratio between the peptide in the sample and the labelled internal standard (“A”) is replaced to the “Y” value in the formula. The concentration of peptide corresponds to the “X” value.

The amount of P1 peptide and its fragments present in the sample were thus determined. The amount of P1 peptide and its fragments corresponds to the amount of unfolded p53 protein (Up53) in the sample. The sample from the test subject was found to contain greater than 0.071 femtomoles/microliter of unfolded p53 protein which shows that the subject from which the sample was taken has a high risk of progressing into Alzheimer's disease within 5-6 years of sample testing.

Thus, the amount of resulting peptide P1 and its fragments that are uniquely present in the samples from a subject having no symptoms or having MCI can be used to diagnose the presence of AD and/or the risk of the subject for progressing into AD.

### INCORPORATION BY REFERENCE

All publications and patents mentioned herein are hereby incorporated by reference in their entirety as if each individual publication or patent was specifically and individually indicated to be incorporated by reference.

### OTHER EMBODIMENTS

While specific embodiments of the subject matter have been discussed, the above specification is illustrative and not restrictive. Many variations will become apparent to those skilled in the art upon review of this specification and the claims below. The full scope of the invention should be determined by reference to the claims, along with their full scope of equivalents, and the specification, along with such variations.

The following are exemplary claims directed to the subject matter described above and should not be considered to limit the present invention; Applicant reserves the right to pursue claims to any of the disclosed subject matter:

1. A method of quantifying an isoform of p53 protein in a biological sample of a subject, said isoform being conformationally altered with respect to the wild-type p53 protein, the method comprising the steps of:
contacting said biological sample or a portion thereof of said subject with an anti-p53 antibody comprising heavy chain CDR sequences (CDR1 (SEQ ID NO:8), CDR2 (SEQ ID NO: 9) and CDR3 (SEQ ID NO: 10)) and light chain CDR sequences (CDR1 (SEQ ID NO:11), CDR2 (SEQ ID NO: 12) and CDR3 (SEQ ID NO: 13)) in a reaction mixture to form an immunocomplex comprising said p53 isoform and said antibody;
eluting p53 isoform to provide eluted p53 isoform;
subjecting said eluted p53 isoform or a portion thereof to enzymatic digestion, thereby to generate a composition comprising one or more proteolytic peptides comprising P1 peptide (TEEENLR, SEQ ID NO: 1);
quantifying an amount of said P1 peptide in said composition or a portion thereof by mass spectrometry, wherein said quantifying comprises determining the intensity of mass/charge ratio corresponding to at least one fragment of said P1 peptide to provide a quantified amount of P1 peptide,
wherein a mass to charge ratio (m/z) of 445 identifies P1 peptide, and wherein a mass to charge ratio selected from the group consisting of: m/z 660, m/z 231, m/z 531, m/z 402, m/z 288, and m/z 358 identifies a fragment of P1 peptide:
wherein said m/z 660 identifies a P1 fragment consisting of amino acids EENLR,
said m/z 231 identifies a P1 fragment consisting of amino acids TE,
said m/z 531 identifies a P1 fragment consisting of amino acids ENLR,
said m/z 402 identifies a P1 fragment consisting of amino acids NLR,
said m/z 288 identifies a P1 fragment consisting of amino acids LR, and
said m/z 358 identifies a P1 fragment consisting of amino acids LR doubly charged; and
wherein said quantified amount of said P1 peptide is proportionate to the amount of said p53 isoform in said sample or portion of said sample.

2. A method to identify in a subject the presence of Alzheimer's disease or prognosis of cognitive decline leading to dementia, the method comprising the steps of:
providing a biological sample of said subject;
contacting the sample or a portion thereof with an antibody that binds an isoform of p53 antibody that is conformationally altered with respect to the wild-type p53 and comprising heavy chain CDR sequences (CDR1 (SEQ ID NO:8), CDR2 (SEQ ID NO: 9) and CDR3 (SEQ ID NO: 10)) and light chain CDR sequences (CDR1 (SEQ ID NO:11), CDR2 (SEQ ID NO: 12) and CDR3 (SEQ ID NO: 13)), wherein the presence of said isoform of p53 protein in said sample permits formation of an immunocomplex comprising said p53 isoform and said antibody;
eluting said p53 isoform to provide eluted p53 isoform;
subjecting said eluted p53 isoform or a portion thereof to enzymatic digestion, thereby to generate a composition comprising one or more proteolytic peptides comprising P1 peptide (TEEENLR, SEQ ID NO: 1);
quantifying an amount of said P1 peptide in said composition or a portion thereof by mass spectrometry, wherein said quantifying comprises determining the intensity of mass/charge ratio corresponding to at least one fragment of said P1 peptide to provide said quantified amount of P1 peptide,
wherein a mass to charge ratio (m/z) of 445 identifies P1 peptide, and wherein a mass to charge ratio selected from the group consisting of: m/z 660, m/z 231, m/z 531, m/z 402, m/z 288, and m/z 358 identifies a fragment of P1 peptide:
wherein said m/z 660 identifies a P1 fragment consisting of amino acids EENLR,
said m/z 231 identifies a P1 fragment consisting of amino acids TE,
said m/z 531 identifies a P1 fragment consisting of amino acids ENLR,
said m/z 402 identifies a P1 fragment consisting of amino acids NLR,
said m/z 288 identifies a P1 fragment consisting of amino acids LR, and
said m/z 358 identifies a P1 fragment consisting of amino acids LR doubly charged; and
wherein when said quantified amount of P1 peptide is proportionate to the amount of said p53 isoform in said sample or portion of said sample, and wherein when said p53 isoform in said sample is higher than a control value, the presence in said subject of Alzheimer's disease or prognosis of cognitive decline leading to dementia is thereby indicated.

3. A method to provide a composition of one or more fragments of P1 peptide (TEEENLR, SEQ ID NO: 1) comprising one or more mass to charge ratio(s) (m/z) selected from the group consisting of 660, 531, 402, 358, 288, and 231, the method comprising the steps of:
contacting a biological sample of a test subject with an anti-p53 isoform antibody comprising heavy chain CDR sequences (CDR1 (SEQ ID NO:8), CDR2 (SEQ ID NO: 9) and CDR3 (SEQ ID NO: 10)) and light chain CDR sequences (CDR1 (SEQ ID NO:11), CDR2 (SEQ ID NO: 12) and CDR3 (SEQ ID NO: 13)) in a reaction mixture, wherein the presence of p53 isoform in said sample permits formation of an immunocomplex comprising said p53 isoform and said antibody, wherein said p53 isoform is conformationally altered with respect to the wild-type p53 protein;
eluting said p53 isoform to provide eluted p53 isoform;
subjecting said eluted p53 isoform or a portion thereof to enzymatic digestion, thereby to generate one or more proteolytic peptides comprising a P1 peptide (TEEENLR, SEQ ID NO: 1);
performing mass spectrometry on said P1 peptide thereby generating said one or more fragments of P1 peptide,
wherein a mass to charge ratio selected from the group consisting of: m/z 660, m/z 231, m/z 531, m/z 402, m/z 288, and m/z 358 identifies a fragment of P1 peptide:
wherein said m/z 660 identifies a P1 fragment consisting of amino acids EENLR,
said m/z 231 identifies a P1 fragment consisting of amino acids TE,
said m/z 531 identifies a P1 fragment consisting of amino acids ENLR,
said m/z 402 identifies a P1 fragment consisting of amino acids NLR,
said m/z 288 identifies a P1 fragment consisting of amino acids LR, and
said m/z 358 identifies a P1 fragment consisting of amino acids LR doubly charged,
and wherein said composition of one or more fragments of P1 peptide optionally comprises a mass to charge ratio (m/z) of 445 that identifies P1 peptide.

4. A method of generating a mass spectrum indicating the presence of P1 peptide (TEEENLR (SEQ ID NO: 1)), the method comprising the steps of:
providing a biological sample of a subject
contacting said biological sample or a portion thereof with an anti p53 isoform antibody in a reaction mixture, said antibody comprising heavy chain CDR sequences (CDR1 (SEQ ID NO:8), CDR2 (SEQ ID NO: 9) and CDR3 (SEQ ID NO: 10)) and light chain CDR sequences (CDR1 (SEQ ID NO:11), CDR2 (SEQ ID NO: 12) and CDR3 (SEQ ID NO: 13)) to form an immunocomplex comprising said p53 isoform and said antibody said isoform being conformationally altered with respect to the wild-type p53 protein;
eluting said p53 isoform to provide eluted p53 isoform;
subjecting said eluted p53 isoform or a portion thereof to enzymatic digestion to generate a composition comprising one or more proteolytic peptides comprising P1 peptide;
subjecting said P1 peptide of said composition to mass spectrometry to thereby generate a mass spectrum,
wherein said mass spectrum comprises at least one mass to charge ratio selected from the group consisting of: 660, m/z 231, m/z 531, m/z 402, m/z 288, and m/z 358, said group optionally including m/z 445;
wherein said m/z 660 identifies a P1 fragment consisting of amino acids EENLR,
said m/z 231 identifies a P1 fragment consisting of amino acids TE,
said m/z 531 identifies a P1 fragment consisting of amino acids ENLR,
said m/z 402 identifies a P1 fragment consisting of amino acids NLR,
said m/z 288 identifies a P1 fragment consisting of amino acids LR, and
said m/z 358 identifies a P1 fragment consisting of amino acids LR doubly charged,
wherein said mass spectrum comprising said at least one mass to charge ratio selected from said group indicates the presence of said P1 peptide fragment in said composition, and wherein said mass spectrum optionally comprises said m/z 445 that identifies P1 peptide consisting of amino acids TEEENLR.

5. The method according to claim 1, wherein said method further comprises a step of quantifying an amount of said P1 peptide, wherein said quantifying comprises determining the intensity of the mass/charge ratio corresponding to at least one fragment of said P1 peptide to provide said quantified amount of P1 peptide, wherein said quantified amount is higher than a control peptide.

6. The method according to any one of claims 1-5, wherein said subject is human.

7. The method according to anyone of claims 1-6, wherein said mass spectrometry is performed in an ion trap mass spectrometer and said at least one P1 peptide fragment comprises a fragment having m/z of 660.

8. The method according to anyone of claims 1-6, wherein said mass spectrometry is performed in an ion trap mass spectrometer and said at least one P1 peptide fragment comprises a fragment having m/z of 531.

9. The method according to anyone of claims 1-6, wherein said mass spectrometry is performed in an ion trap mass spectrometer and said at least one P1 peptide fragment comprises a fragment having m/z of 402.

10. The method according to anyone of claims 1-6, wherein said mass spectrometry is performed in an ion trap mass spectrometer and said at least one P1 peptide fragment comprises a fragment having m/z of 231.

11. The method according to anyone of claims 1-6, wherein said mass spectrometry is performed in an ion trap mass spectrometer or a triple quad mass spectrometer and said P1 peptide fragment comprises a fragment having m/z of 660 and a fragment having m/z of 231.

12. The method according to claim 5, wherein said control peptide is a labeled control peptide.

13. The method according to claim 12, wherein said labeled control peptide is a labeled P1 peptide and is preferably internal to said composition and is represented as a value comprising its concentration in said reaction mixture.

14. The method according to anyone of claims 1-6, wherein said biological sample is subjected to protein plasma depletion prior to said contacting.

15. The method of according to claim 14, wherein said protein plasma depletion is accomplished by one or more of: HPLC, a chromatographic column, and/or chemical treatment of said biological sample.

16. The method according to claim 2, wherein said subject is an asymptomatic subject or exhibits mild cognitive impairment.

17. The method according to anyone of claims 1-6, wherein said mass spectrometry is performed in an ion trap mass spectrometer or a triple quad mass spectrometer and said at least one P1 peptide fragment comprises two fragments having m/z of 660 and m/z of 231.

18. The method according to anyone of claims 1-6, wherein said biological sample is blood, plasma, serum, saliva, urine, neuronal cells, or blood cells.

19. The method according to any one of claims 1-6, wherein said mass spectrometry is performed in a mass spectrometer and said mass spectrometer is one of MALDI-MS, Ion trap mass spectrometer, Linear ion trap mass spectrometer, Kindon trap mass spectrometer, Paul trap mass spectrometer, Orbitrap mass spectrometer, Quadrupole ion trap mass spectrometer, Triple quadruple ion trap mass spectrometer, and Ion cyclotron residence trap mass spectrometer.

20. The method according to any one of claims 1-19, wherein said antibody is a monoclonal antibody.

21. The method according to anyone of claims 1-6, wherein said mass spectrometry is performed in a triple quad mass spectrometer and said at least one P1 peptide fragment has an m/z of 660.

22. The method according to anyone of claims 1-6, wherein said mass spectrometry is performed in a triple quad mass spectrometer and said at least one P1 peptide fragment comprises has an m/z of 531.

23. The method according to anyone of claims 1-6, wherein said mass spectrometry is performed in a triple quad mass spectrometer and said at least one P1 peptide fragment comprises a fragment having an m/z of 402.

24. The method according to anyone of claims 1-6, wherein said mass spectrometry is performed in an ion trap mass spectrometer or a triple quad mass spectrometer and said at least one P1 peptide fragment comprises a fragment having an m/z of 288.

25. The method according to anyone of claims 1-6, wherein said mass spectrometry is performed in a triple quad mass spectrometer and said at least one P1 peptide fragment comprises a fragment having an m/z of 231.

26. The method according to anyone of claims 1-6, wherein said mass spectrometry is performed in an ion trap mass spectrometer or a triple quad mass spectrometer and said P1 peptide fragment comprises a fragment having an m/z of 231 and a fragment having an m/z of 445.

27. The method of claim 1, wherein a higher amount of said P1 peptide relative to a control value indicates Alzheimer's disease or cognitive decline leading to dementia in said subject.

28. The method of anyone of claims 2 or 27, wherein said higher amount of said P1 peptide is greater than 0.071 femtomoles per microliter.

29. The method of anyone of claims 2 or 27, wherein said method further comprises the step of treating Alzheimer's disease or cognitive decline leading to dementia using chemical or biological drugs.

30. The method of claim 29, wherein said chemical drugs are selected from the group consisting of selective serotonin reuptake inhibitor, donepezil, galantamine, rivastigmine, suvorexant, brexpiprazole and memantine.

31. The method of claim 29, wherein said biological drugs are selected from the group consisting of Aducanumab and Lecanemab.

32. The method of according to claims 1-2 and 5-30, wherein said quantifying further comprises determining the intensity of the mass/charge ratio (m/z) corresponding to said P1 peptide and combining it with the intensity of m/z corresponding to said at least one fragment.

33. The method of according to claims 1-2 and 5-30, wherein said quantifying further comprises determining the intensity of m/z corresponding to said P1 peptide and combining it with the intensity of mass/charge ratio corresponding to at least two fragments.

34. The method of according to claim 33, wherein said quantifying further comprises determining the intensity of m/z corresponding to said P1 peptide and combining it with the intensity of mass/charge ratio corresponding to at least three fragments.

35. The method of according to claim 34, wherein said quantifying further comprises determining the intensity of m/z corresponding to said P1 peptide and combining it with the intensity of mass/charge ratio corresponding to at least four fragments.

36. The method of according to claim 1 or 2, wherein said quantifying comprises determining the intensity of m/z corresponding to at least two fragments of said P1 peptide having m/z selected from the group consisting of 660, 231, 531, 402, and 288.


SEQUENCE LISTING (24 sequences)

SEQ ID NO: 1 - protein, 16 aa, synthetic construct
  REGION 1..16: note = Linear Epitope
RRTEEENLRKKGEPHH

SEQ ID NO: 2 - protein, 16 aa, synthetic construct
  REGION 1..16: note = Immunization Peptide
CRTEEENLRKKGEPHH

SEQ ID NO: 3 - protein, 392 aa, synthetic construct
  REGION 1..392: note = p53 Protein
MEEPQSDPSVEPPLSQETFSDLWKLLPENNVLSPLPSQAMDDLMLSPDDIEQWFTEDPGPDEAPRMPEAAPPVAPAPAAPTPAAPAPAPSWPLSSSVPSQKTYQGSYGFRLGFLHSGTAKSVTCTYSPALNKMFCQLAKTCPVQLWVDSTPPPGTRVRAAIYKQSQHMTEVVRRCPHHERCSDSDGLAPPQHLIRVEGNLRVEYLDDRNTFRHSVVVPYEPPEVGSDCTTIHYNYMCNSSCMGGMNRRPILTIITLEDSSGNLLGRNSFEVRVCACPGRDRRTEEENLRKKGEPHHELPPGSTKRALPNNTSSSPQPKKKPLDGEYFTLQIRGRERFEMFRELNEALELKDAQAGKEPGGSRAHSSHLKSKKGQSTSRHKKLMFKTEGPDSD

SEQ ID NO: 4 - protein, 572 aa, synthetic construct
  REGION 1..572: note = Heavy chain of 2D3A8 antibody
EVQLQQSGPELVKPGASVKMSCKASGYTFTSYVMHWVKQKPGQGLEWIGYINPYNDGTKYNEKFKGKATLTSDKSSSTAYMELSSLTSEDSAVYYCARGGYYAMDYWGQGTSVTVSSESQSFPNVFPLVSCESPLSDKNLVAMGCLARDFLPSTISFTWNYQNNTEVIQGIRTFPTLRTGGKYLATSQVLLSPKSILEGSDEYLVCKIHYGGKNRDLHVPIPAVAEMNPNVNVFVPPRDGFSGPAPRKSKLICEATNFTPKPITVSWLKDGKLVESGFTTDPVTIENKGSTPQTYKVISTLTISEIDWLNLNVYTCRVDHRGLTFLKNVSSTCAASPSTDILTFTIPPSFADIFLSKSANLTCLVSNLATYETLNISWASQSGEPLETKIKIMESHPNGTFSAKGVASVCVEDWNNRKEFVCTVTHRDLPSPQKKFISKPNEVHKHPPAVYLLPPAREQLNLRESATVTCLVKGFSPADISVQWLQRGQLLPQEKYVTSAPMPEPGAPGFYFTHSILTVTEEEWNSGETYTCVVGHEALPHLVTERTVDKSTGKPTLYNVSLIMSDTGGTCY

SEQ ID NO: 5 - protein, 214 aa, unidentified
  REGION 1..214: note = Light chain of 2D3A8 antibody
DIQMTQTTSSLSASLGDRVTISCRASQDISNYLNWYQQKPDGTVKLLIYYTSRLHSGVPSRFSGSGSGTDYSLTISNLEQEDIATYFCQQGNTLPYTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 6 - protein, 117 aa, synthetic construct
  REGION 1..117: note = Heavy chain variable region of 2D3A8 antibody
EVQLQQSGPELVKPGASVKMSCKASGYTFTSYVMHWVKQKPGQGLEWIGYINPYNDGTKYNEKFKGKATLTSDKSSSTAYMELSSLTSEDSAVYYCARGGYYAMDYWGQGTSVTVSS

SEQ ID NO: 7 - protein, 107 aa, synthetic construct
  REGION 1..107: note = Light chain variable region of 2D3A8 antibody
DIQMTQTTSSLSASLGDRVTISCRASQDISNYLNWYQQKPDGTVKLLIYYTSRLHSGVPSRFSGSGSGTDYSLTISNLEQEDIATYFCQQGNTLPYTFGGGTKLEIK

SEQ ID NO: 8 - protein, 5 aa, synthetic construct
  REGION 1..5: note = Heavy chain CDR1 of 2D3A8 antibody
SYVMH

SEQ ID NO: 9 - protein, 17 aa, synthetic construct
  REGION 1..17: note = Heavy chain CDR2 of 2D3A8 antibody
YINPYNDGTKYNEKFKG

SEQ ID NO: 10 - protein, 8 aa, synthetic construct
  REGION 1..8: note = Heavy chain CDR3 of 2D3A8 antibody
GGYYAMDY

SEQ ID NO: 11 - protein, 11 aa, synthetic construct
  REGION 1..11: note = Light chain CDR1 of 2D3A8 antibody
RASQDISNYLN

SEQ ID NO: 12 - protein, 7 aa, synthetic construct
  REGION 1..7: note = Light chain CDR2 of 2D3A8 antibody
YTSRLHS

SEQ ID NO: 13 - protein, 9 aa, synthetic construct
  REGION 1..9: note = Light chain CDR3 of 2D3A8 antibody
QQGNTLPYT

SEQ ID NO: 14 - protein, 7 aa, synthetic construct
  REGION 1..7: note = P1 peptide
TEEENLR

SEQ ID NO: 15 - protein, 5 aa, synthetic construct
  REGION 1..5: note = P1 fragment
EENLR

SEQ ID NO: 16 - skipped

SEQ ID NO: 17 - protein, 4 aa, synthetic construct
  REGION 1..4: note = P1 fragment
ENLR

SEQ ID NO: 18-19 - skipped (2 entries)

SEQ ID NO: 20 - protein, 5 aa, synthetic construct
  REGION 1..5: note = P1 fragment
TEEEN

SEQ ID NO: 21 - skipped

SEQ ID NO: 22 - protein, 6 aa, synthetic construct
  REGION 1..6: note = P1 fragment
EEENLR

SEQ ID NO: 23 - protein, 4 aa, synthetic construct
  REGION 1..4: note = P1 fragment
TEEE

SEQ ID NO: 24 - skipped